(19) United States
(12) Patent Application Publication
(10) Pub. No.: US 2013/0217014 A1
(43) Pub. Date: Aug. 22, 2013

(54) METHODS OF USING CDK8 ANTAGONISTS

(71) Applicant: GENENTECH, INC., (US)

(72) Inventors: Ron Firestein, Burlingame, CA (US); Adam Shultz, Emerald Hills, CA (US)

(73) Assignee: GENENTECH, INC., South San Francisco, CA (US)

(21) Appl. No.: 13/769,105

(22) Filed: Feb. 15, 2013

Related U.S. Application Data
(60) Provisional application No. 61/600,539, filed on Feb. 17, 2012.

(51) Int. Cl. G01N 33/50; C12Q 1/68; C12N 5/095

(52) U.S. Cl. CPC G01N 33/5073; C12N 5/0695; C12Q 1/6886
USPC 435/6.11; 435/7.1; 435/6.1; 435/377; 435/6.13; 506/9

(57) ABSTRACT

Provided herein are CDK8 antagonists and methods of using the same.

### CROSS REFERENCE TO RELATED APPLICATIONS

This application claims benefit under 35 USC 119(e) to provisional application No. 61/600,539 filed Feb. 17, 2012, the contents of which are incorporated herein in their entirety by reference.

### FIELD

The present invention relates to methods of inducing differentiation, particularly differentiation of tumor cells, by inhibition of CDK8.

### BACKGROUND

Recent studies have highlighted the ability of tumors to employ genetic programs normally found active in the embryonic state. Young, R. A., Cell 144, 940-954 (2011); Takebe, N. et al., Nat Rev Clin Oncol 8, 97-106 (2011); Ben-Porath, I. et al., Nat Genet. 40, 499-507 (2008); Wong D. J. et al., Cell Stem Cell 2:333-344 (2008). In embryonic stem cells, pluripotency-related gene signatures are often re-expressed in multiple cancer types, and expression of these stem cell-related genes strongly correlate with poor clinical outcome. Ben-Porath, I. et al., Nat Genet. 40, 499-507 (2008); Wong D. J. et al., Cell Stem Cell 2:333-344 (2008). Identifying new druggable targets that are critical to the stem cell-like properties of tumors offers a new avenue of therapeutic intervention. The MYC oncogene is a critical transcriptional regulator in many tumor types (Meyer N. & Penn L. Z. Nat Rev Cancer 8:976-90 (2008)) and has also been demonstrated to play an essential role in ES cell proliferation and pluripotency but has been an intractable therapeutic target. Young, R. A., Cell 144, 940-954 (2011); Cartwright P. et al., Development 132:885-96 (2005).

CDK8 is a cyclin dependent kinase that has a conserved function in transcription as part of the Mediator complex. Taatjes, D. J., Trends Biochem Sci 35, 315-322 (2010); Conaway, R. C. and Conaway, J. W., Curr Opin Genet Dev 21, 225-230 (2011). More recently, CDK8 has been reported to as an oncogene in both colon cancer (Firestein R. et al., Nature 455:547-51 (2008); Morris E. J. et al., Nature 455:552-6 (2008); Starr T. K. et al., Science 323:1747-50 (2009)) and melanoma (Kapoor A. et al., Nature 468:1105-9 (2010)). CDK8 is upregulated and amplified in a subset of human colon tumors. CDK8 transforms immortalized cells and is required for colon cancer proliferation in vitro. Firestein, R. et al., Nature 455, 547-551 (2008). CDK8 has also been found to be overexpressed and essential for proliferation in melanoma. Kapoor, A. et al., Nature 468, 1105-1109 (2010). CDK8 has been shown to regulate several signaling pathways that are key regulators of both ES pluripotency and cancer. CDK8 activates the Wnt pathway by promoting expression of β-Catenin target genes (Firestein, R. et al., Nature 455, 547-551 (2008)) or by inhibiting E2F1, a potent inhibitor of β-Catenin transcriptional activity. Morris, E. J. et al., Nature 455, 552-556 (2008). CDK8 promotes Notch target gene expression by phosphorylating the Notch intracellular domain, activating Notch enhancer complexes at target genes. Fryer C. J. et al., Mol Cell 16:509-20 (2004). Lastly, CDK8 phosphorylation of SMAD proteins leads to activation of TGF-β/BMP target genes followed by degradation of the SMAD proteins to limit the target gene expression. Alarcon, C. et al., Cell 139, 757-769 (2009). Many of these studies, however, were conducted in vitro, in cell based assays that miss certain aspects of tumor growth in vivo.

There is a need to understand the functional and molecular consequences of CDK8 loss in both fully formed tumors and ES cells and better predict clinical outcome and differentiation status in colon cancer patients.

### SUMMARY

The invention provides CDK8 antagonist and methods of using the same. Provided herein methods of screening for and/or identifying a CDK8 antagonist which promotes cell differentiation said method comprising: contacting a reference cell, wherein the reference cell is a stem cell and/or a cancer stem cell, with a CDK8 candidate antagonist, wherein the CDK8 candidate antagonist binds CDK8, and whereby differentiation of the reference cell into a differentiated cell identifies the CDK8 candidate antagonist as a CDK8 antagonist which promotes cell differentiation. In some embodiments, the reference cell is a cancer stem cell. In some embodiments, the differentiated cell is a goblet cell and/or enterocyte cell. In some embodiments, the CDK8 candidate antagonist is an antibody, binding polypeptide, small molecule, or polynucleotide.

In another aspect, provided herein are methods of inducing differentiation comprising contacting the cell with an effective amount of CDK8 antagonist. In some embodiments, the cell is a stem cell. In some embodiments, the cell is a cancer stem cell.

Provided herein are methods of treating a cancer cell, wherein the cancer cell differentially expresses one or more biomarkers of a CDK8 gene signature (e.g., compared to a reference sample, reference cell, reference tissue, control sample, control cell, control tissue, or internal control (e.g., housekeeping gene)), the method comprising providing an effective amount of a CDK8 antagonist. In some embodiments, differential expression of one or more biomarkers of the CDK8 gene signature is elevated expression of one or more CDK8-induced biomarkers of the CDK8 gene signature and/or reduced expression of one or more CDK8-repressed biomarkers of the CDK8 gene signature.

Further provided herein are methods of treating cancer in an individual comprising administering to the individual an effective amount of a CDK8 antagonist, wherein treatment is based upon the cancer comprising cancer stem cell-like properties. In some embodiments, the cancer stem cell-like properties comprise differential expression levels of one or more biomarkers of a CDK8 gene signature in a sample from the individual (e.g., compared to a reference sample, reference cell, reference tissue, control sample, control cell, control tissue, or internal control (e.g., housekeeping gene)). In some embodiments, differential expression of one or more biomarkers of the CDK8 gene signature is elevated expression of one or more CDK8-induced biomarkers of the CDK8 gene signature and/or reduced expression of one or more CDK8-repressed biomarkers of the CDK8 gene signature.

Further provided herein are methods of treating a disease or disorder in an individual comprising administering to the individual an effective amount of a CDK8 antagonist, wherein treatment is based upon differential expression levels of one or more biomarkers of a CDK8 gene signature in a sample from the individual (e.g., compared to a reference sample, reference cell, reference tissue, control sample, control cell, control tissue, or internal control (e.g., housekeeping gene)). In some embodiments, differential expression of one or more biomarkers of the CDK8 gene signature is elevated expression of one or more CDK8-induced biomarkers of the CDK8 gene signature and/or reduced expression of one or more CDK8-repressed biomarkers of the CDK8 gene signature.

Provided herein are methods of treating a disease or disorder in an individual comprising administering to the individual an effective amount of a CDK8 antagonist, wherein treatment is continued based upon differential expression levels of one or more biomarkers of a CDK8 gene signature in a sample from the individual (e.g., compared to a reference sample, reference cell, reference tissue, control sample, control cell, control tissue, or internal control (e.g., housekeeping gene)). In some embodiments, differential expression of one or more biomarkers of the CDK8 gene signature is reduced expression of one or more CDK8-induced biomarkers of the CDK8 gene signature and/or elevated expression of one or more CDK8-induced biomarkers of the CDK8 gene signature.

Further provided herein are methods for treating a disease or disorder in an individual, the method comprising: determining that a sample obtained from the individual comprises differential expression levels of one or more biomarkers of a CDK8 gene signature (e.g., compared to a reference sample, reference cell, reference tissue, control sample, control cell, control tissue, or internal control (e.g., housekeeping gene)), and administering an effective amount of a CDK8 antagonist to the individual, whereby the disease or disorder is treated. In some embodiments, differential expression of one or more biomarkers of the CDK8 gene signature is elevated expression of one or more CDK8-induced biomarkers of the CDK8 gene signature and/or reduced expression of one or more CDK8-repressed biomarkers of the CDK8 gene signature.

Provided herein are methods of treating disease or disorder in an individual, comprising: (a) selecting an individual having differential expression levels of one or more biomarkers of a CDK8 gene signature in a sample from the individual (e.g., compared to a reference sample, reference cell, reference tissue, control sample, control cell, control tissue, or internal control (e.g., housekeeping gene)); and (b) administering to the individual thus selected an effective amount of a CDK8 antagonist, whereby the disease or disorder is treated. In some embodiments, differential expression of one or more biomarkers of the CDK8 gene signature is elevated expression of one or more CDK8-induced biomarkers of the CDK8 gene signature and/or reduced expression of one or more CDK8-repressed biomarkers of the CDK8 gene signature.

Also provided herein are methods of identifying an individual with a disease or disorder who is more or less likely to exhibit benefit from treatment with a therapy comprising a CDK8 antagonist, the method comprising: determining the expression levels of one or more biomarkers of a CDK8 gene signature in a sample from the individual, wherein differential expression levels of one or more biomarkers of the CDK8 gene signature in a sample from the individual (e.g., compared to a reference sample, reference cell, reference tissue, control sample, control cell, control tissue, or internal control (e.g., housekeeping gene)) indicates that the individual is more likely to exhibit benefit from treatment with the therapy comprising the CDK8 antagonist and/or non-differential expression levels of one or more biomarkers of the CDK8 gene signature in a sample from the individual (e.g., compared to a reference sample, reference cell, reference tissue, control sample, control cell, control tissue, or internal control (e.g., housekeeping gene)) indicates that the individual is less likely to exhibit benefit from treatment with the therapy comprising the CDK8 antagonist. In some embodiments, the method further comprises administering an effective amount of a therapy comprising a CDK8 antagonist. In some embodiments, differential expression of one or more biomarkers of the CDK8 gene signature is elevated expression of one or more CDK8-induced biomarkers of the CDK8 gene signature and/or reduced expression of one or more CDK8-repressed biomarkers of the CDK8 gene signature.

Provided herein are methods for predicting whether an individual with a disease or disorder is more or less likely to respond effectively to treatment with a therapy comprising a CDK8 antagonist, the method comprising assessing expression levels of one or more biomarkers of a CDK8 gene signature in a sample from the individual, whereby differential expression levels of one or more biomarkers of the CDK8 gene signature in a sample from the individual (e.g., compared to a reference sample, reference cell, reference tissue, control sample, control cell, control tissue, or internal control (e.g., housekeeping gene)) indicates that the individual is more likely to respond effectively to treatment with the CDK8 antagonist and/or non-differential expression levels of one or more biomarkers of the CDK8 gene signature in a sample from the individual (e.g., compared to a reference sample, reference cell, reference tissue, control sample, control cell, control tissue, or internal control (e.g., housekeeping gene)) indicates that the individual is less likely to respond effectively to treatment with the CDK8 antagonist. In some embodiments, the method further comprises administering an effective amount of a therapy comprising a CDK8 antagonist. In some embodiments, differential expression of one or more biomarkers of the CDK8 gene signature is elevated expression of one or more CDK8-induced biomarkers of the CDK8 gene signature and/or reduced expression of one or more CDK8-repressed biomarkers of the CDK8 gene signature.

Provided herein are methods of predicting the response or lack of response of an individual with a disease or disorder to a therapy comprising a CDK8 antagonist comprising measuring expression levels of one or more biomarkers of a CDK8 gene signature in a sample from the individual, wherein differential expression levels of one or more biomarkers of the CDK8 gene signature in a sample from the individual (e.g., compared to a reference sample, reference cell, reference tissue, control sample, control cell, control tissue, or internal control (e.g., housekeeping gene)) is predictive of response of the individual to the therapy comprising the CDK8 antagonist and non-differential expression levels of one or more biomarkers of the CDK8 gene signature in a sample from the individual (e.g., compared to a reference sample, reference cell, reference tissue, control sample, control cell, control tissue, or internal control (e.g., housekeeping gene)) is predictive of lack of response of the individual to the therapy comprising the CDK8 antagonist. In some embodiments, the method further comprises administering an effective amount of a therapy comprising a CDK8 antagonist. In some embodiments, differential expression of one or more biomarkers of the CDK8 gene signature is elevated expression of one or more CDK8-induced biomarkers of the CDK8 gene signature and/or reduced expression of one or more CDK8-repressed biomarkers of the CDK8 gene signature.

Further provided herein are methods of determining whether an individual having a disease or disorder is more or less likely responding to therapy, wherein therapy comprises a CDK8 antagonist, based upon levels of one or more biomarkers of a CDK8 gene signature in a sample from the individual, wherein differential expression levels of one or more biomarkers of the CDK8 gene signature in a sample from the individual (e.g., compared to a reference sample, reference cell, reference tissue, control sample, control cell, control tissue, or internal control (e.g., housekeeping gene)) identifies the individual as more likely responding to therapy comprising the CDK8 antagonist and non-differential expression levels of one or more biomarkers of the CDK8 gene signature in a sample from the individual (e.g., compared to a reference sample, reference cell, reference tissue, control sample, control cell, control tissue, or internal control (e.g., housekeeping gene)) identifies the individual as less likely responding to therapy comprising the CDK8 antagonist. In some embodiments, the method further comprises administering an effective amount of a therapy comprising a CDK8 antagonist. In some embodiments, differential expression of one or more biomarkers of the CDK8 gene signature is reduced expression of one or more CDK8-induced biomarkers of the CDK8 gene signature and/or elevated expression of one or more CDK8-induced biomarkers of the CDK8 gene signature.

In some embodiments of any of the methods, the one or more biomarkers of the CDK8 gene signature comprises one or more biomarkers of the CDK8 cancer cell gene signature. In some embodiments, the one or more biomarkers of the CDK8 cancer cell gene signature comprises one or more genes listed in Table 2. In some embodiments, the one or more genes listed in Table 2 comprises one or more ES cell-related genes, MYC ES target genes, p53 signalling genes, cell cycle genes, Wnt signalling genes, and/or SMAD/BMP signalling genes.

In some embodiments of any of the methods, the one or more biomarkers of the CDK8 gene signature comprises one or more biomarkers of the CDK8 embryonic stem cell gene signature. In some embodiments, the one or more biomarkers of the CDK8 embryonic stem cell gene signature comprises one or more genes listed in Table 3.

In some embodiments of any of the methods, the disease or disorder is cancer.

In some embodiments of any of the methods, the CDK8 antagonist is an antibody, binding polypeptide, small molecule, or polynucleotide. In some embodiments, the CDK8 antagonist is an antibody. In some embodiments, the CDK8 antagonist is a small molecule. In some embodiments, the small molecule is a small molecule kinase inhibitor. In some embodiments, the small molecule kinase inhibitor is selected from the group consisting of flavopiridol, ABT-869, AST-487, BMS-387032/SNS032, BIRB-796, sorafenib, staurosporine, cortistatin, cortistatin A, and/or a steroidal alkaloid or derivative thereof. In some embodiments, the CDK8 antagonist induces cell cycle arrest or is capable of promoting differentiation. In some embodiments, wherein the CDK8 antagonist is capable of promoting a change in cell fate and promoting differentiation is indicated by reduced expression of one or more CDK8-induced biomarkers of the CDK8 gene signature and/or elevated expression of one or more CDK8-reduced biomarkers of the CDK8 gene signature.

### BRIEF DESCRIPTION OF THE FIGURES

FIG. 1|CDK8 was required for tumor growth and maintenance of a de-differentiated state in vivo. A Xenograft tumor volume measurements over time (n=10 mice per group). The tumor growth inhibition values were determined by an area under the curve calculation. Mean±s.e.m. is shown *, P=0.001 compared to all other groups, Student's t-test. B Western blot of CDK8 protein levels in shCDK8 xenograft tumors at Day 8 (HT-29) or Day 12 (COLO 205) (top) Immunohistochemistry of CDK8 protein in HT-29 shCDK8 tumors from the end of the study (bottom). C Images of hematoxylin and eosin (H&E) stained tumors from the end of the study. HT-29 shCDK8 tumors were stained with alcian blue that stains secreted mucin. Asterisks indicate the lumen of well-formed glands seen in COLO 205 shCDK8 tumors. D Quantitative RT-PCR and Western blot analysis of CDK8 three days after siRNA transfection in HT-29 human colon cancer cells. Mean±s.d. is shown *, P=10⁻⁵, Student's t-test. E The top 1500 genes that change after CDK8 knockdown in HT-29 cells relative to siNTC (P<0.001, Student's t-test between siNTC and siCDK8-1/-2). GO, gene ontology.

FIG. 2|CDK8 maintained ES cells in an undifferentiated state. A, Images of alkaline phosphatase stained R1 mouse ES cells before and after induced differentiation. Positive staining (red) indicates undifferentiated stem cells. B, C, Quantitative RT-PCR and Western blot detection of NANOG (B) and CDK8 (C) at the indicated times after ES cell differentiation. CDK8 protein levels were quantified relative to ACTIN and normalized to Day 0. Mean±s.d. is shown. *, P=10⁻⁵, Student's t-test. D, Alkaline phosphatase staining and phase contrast images of ES cells at the indicated times following shRNA infection. The staining in shNTC samples was representative of all subsequent time points. The requirement for CDK8 to maintain ES cells in an undifferentiated state was observed in at least three independent knockdown experiments. The number of alkaline phosphatase stained ES cell colonies observed per 24 mm² field is shown to the right. *, P=0.001 compared to shNTC, Student's t-test. E, Western blot of shRNA infected ES cells at Day 13 after infection. F, Alkaline phosphatase staining and phase contrast images of ES cells at Day 11 after exogenous expression of CDK8 or empty vector in the presence of the indicated shRNA. G, Quantitation of alkaline phosphatase stained ES cell colonies observed per 24 mm² field. Mean±s.d. is shown. P=10⁻⁵ between shCdk8+vector and shCdk8+CDK8, Student's t-test. H, Western blot of CDK8-rescued ES cells at Day 11. Exogenously expressed human CDK8 protein was shifted slightly higher on the gel due to it being FLAG-tagged.

FIG. 3|CDK8 regulated MYC target gene and protein levels. A, Shown are the top 1500 genes that change after CDK8 knockdown in R1 ES cells at Day 8 relative to shNTC control (P=0.003, Student's t-test between shNTC and two independent CDK8 shRNAs). The expression of these same genes at Day 13 is shown. B, Quantitative RT-PCR analysis of two representative ES cell genes (H2afx and Tcl1) at the indicated times after shCDK8 treatment. Mean±s.d. is shown. *, P=0.05, Student's t-test. C, Western blot of infected ES cells at the indicated time after shRNA infection. A schematic of common MYC phosphorylation modifications is on the left. D, OCT4, NANOG, and MYC proteins levels were quantified relative to ACTIN, and then normalized to their respective shNTC for each time point. E, For each time point MYC-pS62 and MYC-pT58 protein levels were quantified relative to total MYC and then normalized to their respective shNTC.

FIG. 4|CDK8 partially regulated ES cell pluripotency through MYC. A Western blot of ES cells stably expressing MYC, MYC^{T58A}, MYC^{S62A}, or GFP in the presence of the indicated shRNA at Day 11. Total MYC protein was quantified relative to ACTIN and normalized to their respective shNTC. The anti-MYC antibody detects mouse and human MYC. B, Alkaline phosphatase staining and phase contrast images of ES cells at Day 11 after expression of MYC, MYC^{T58A}, MYC^{S62A}, or GFP control in the presence of the indicated shRNA. The number of alkaline phosphatase stained ES cell colonies observed per 24 mm² field is shown to the right of each group. The dashed gray line indicated the number of colonies observed in shNTC+GFP control cells. Mean±s.d. is shown *, P=0.005 compared to the respective GFP expression control, Student's t-test.

FIG. 5|Coordinated expression of CDK8-regulated MYC targets in primary human colon cancer. A, Gene set enrichments in HT-29 CDK8-induced and CDK8-repressed genes. B, Quantitative RT-PCR of four MYC-driven ES cell target genes in HT-29 cells. Mean±s.d. is shown. *, P=0.01, Student's t-test. C, The log₂ mean centered expression of CDK8-induced MYC ES cell target genes (from FIG. 5A) was shown for 227 primary and metastatic human colon tumors (from Gene Logic). The tumors were sorted based on high to low average expression of the CDK8-induced MYC ES cell targets and split in two at the mean expression level. Bar graph depicted average±s.e.m. log₂ expression of CDK8 in the two groups (P=0.002, Student's t-test). D, Western blot analysis of normal colon, primary colon tumors, and metastatic colon tumors. CDK8 and MYC levels were quantified relative to ACTIN then normalized to their average in normal colon. Phospho-specific MYC (S62 and T58) levels were quantified relative to MYC, then the ratio was normalized to their average ratio in normal colon. P-values for Pearson correlations are one-tailed t-tests. E, The average log₂ expression of the CDK8-induced MYC ES cell targets was sorted high to low for 213 primary human colon tumors with known tumor differentiation status (Smith J J. et al., Gastroenterology 138:958-68 (2010)). Hash marks indicated poorly differentiated tumors; the remaining tumors are either well or moderately differentiated. P-values were calculated with a fisher exact test using a 2×2 contingency table. F, The average log₂ expression of the CDK8-induced MYC ES cell targets for 50 primary human colon tumors that underwent recurrence (Jorissen R. N. et al., Colorectal Cancer. Clin Cancer Res 15:7642-51 (2009).); time to recurrence was indicated below each tumor. The average time to recur±s.e.m. for each group is shown (P=0.02, Student's t-test).

FIG. 6|G ene expression analysis of ES cell-related genes in HT-29 siCDK8 cells. Quantitative RT-PCR of multiple ES cell-related genes three days after CDK8 siRNA transfection in HT-29 human colon cancer cells. Expression was normalized to siNTC treated cells. Mean+/−s.d. is shown. *, P=0.01, Student's t-test).

FIG. 7|CDK8 maintained multiple ES cell lines in an undifferentiated state. A, Western blot of the indicated shRNA infected ES cell lines at Day 7 after infection. B, Alkaline phosphatase staining and phase contrast images at Day 7. The number of alkaline phosphatase stained ES cell colonies observed per 24 mm² filed is shown below. *, P=10⁻⁴, Student's t-test).

FIG. 8|CDK8 and MED12 regulated distinct gene expression programs in ES cells. A, Quantitative RT-PCR of Med12 levels at Day 13 after MED12 shRNA infection in R1 ES cells. Mean+/−s.d. is shown. *, P=10⁻⁶, Student's t-test). B, Alkaline phosphatase staining and phase contrast images of ES cells at Day 13 after MED12 shRNA treatment. C, Shown are the top 1500 genes that changed after CDK8 knockdown at Day 8 in R1 ES cells relative to shNTC control. The expression of these same genes following MED12 knockdown at Day 13 is shown. D, Shown are a set of genes found to be regulated by MED12 in mouse ES cells (Kagey et al., Nature (2010)). Expression data of these genes from the previous study is sorted low to high. Next to this is expression of the same genes from this study in mouse ES cells and from mouse ES cells that have undergone forced differentiation through three different chemical methods or mouse ES cells that have differentiated following siNanog or SiOct4 treatment (data from Gene Expression Omnibus accession GSE4189; Loh et al., Nature Genetics (2006)). The fold change scale for each data set relative to shNTC or siNTC controls is indicated on the right. E, The bar graph shows the Pearson correlations of the gene expression pattern for each indicated data set with the shMed12 expression pattern from Kagey et al. P-values of various Pearson correlations were calculated with one-tailed t-tests.

FIG. 9|A, B, Loss of CDK8 leads to decreased MYC protein level but does not alter its subcellular localization. A, Immunofluorescence images of MYC and CDK8 in R1 ES cells at Day 8 after shRNA infection. Cell nuclei are indicated by Hoechst staining B, Immunofluorescence images of MYC and phosphor-specific MYC proteins in ES cells at Day 13 after shRNA infection. C, Myc expression weakly changes upon CDK8 loss in ES cells. Quantitative RT-PCR analysis of Myc at Day 8 and Day 13 of shCdk8 treatment in R1 ES cells. Mean+/−s.d. is shown. *, P=0.002, Student's t-test).

FIG. 10|Gene expression analysis of MYC ES cell targets in HT-29 siCDK8 cells. Quantitative RT-PCR of multiple MYC ES cell targets (previously identified through chromatin IP experiments in mouse ES cells; Kim et al., Cell (2008)) three days after CDK8 siRNA transfection in HT-29 human colon cancer cells. Expression is normalized to siNTC treated cells. Mean+/−s.d. is shown. *, P=0.01, Student's t-test).

FIG. 11|MYC is co-expressed with the HT-29 CDK8-regulated gene signature in human colon cancer. A, Bar graph shows Pearson correlations of the indicated transcription factor and pathway genes with expression of the CDK8-regulated HT-29 signature (from FIG. 1E) in human tumors (n=230 total). Genes with a positive Pearson correlation indicate that the gene is co-expressed with the CDK8 signature. Dashed lines specify P-value cut-offs for low and high correlations (P-values calculated with one-tailed t-test). B, Correlation of high MYC expression with increased expression of the HT-29 CDK8-regulated signature. Bar graph depicts log₂ mean centered MYC expression for individual human colon tumor samples. Tumors were sorted from high to low based on expression of the CDK8 signature (the dark bar on the left indicates CDK8-induced genes; the grey bar indicates CDK8-repressed genes). Higher MYC expression was seen in tumors that express the CDK8-regulated signature, while low MYC expression is seen in tumors with the opposite pattern of the CDK8 signature (Pearson=0.57, P=10⁻¹², Student's t-test).

### DETAILED DESCRIPTION

### I. Definitions

The terms “CDK8” and “cyclin-dependent kinase 8” refer herein to a native CDK8 from any vertebrate source, including mammals such as primates (e.g., humans) and rodents (e.g., mice and rats), unless otherwise indicated. The term encompasses “full-length,” unprocessed CDK8 as well as any form of CDK8 that results from processing in the cell. The term also encompasses naturally occurring variants of CDK8, e.g., splice variants or allelic variants. The sequence of an exemplary human CDK8 nucleic acid sequence is NM_{—}001260 (gi:4502744) or an exemplary human CDK8 is amino acid sequence of CDK8 NP_{—}001251.1, UniProtKB/Swiss-Prot:P49336, P49336.2, and/or P49336.1.

“CDK8 variant” or variations thereof, means a CDK8 polypeptide or polynucleotide, generally being or encoding an active CDK8 polypeptide, as defined herein having at least about 80% amino acid sequence identity with any of the native sequence CDK8 polypeptide sequences as disclosed herein. Such CDK8 variants include, for instance, CDK8 wherein one or more nucleic acid or amino acid residues are added or deleted. Ordinarily, a CDK8 variant will have at least about 80% sequence identity, alternatively at least about 81%, 82%, 83%, 84%, 85%, 86%, 87%, 88%, 89%, 90%, 91%, 92%, 93%, 94%, 95%, 96%, 97%, 98%, or 99% sequence identity, to CDK8 as disclosed herein. Ordinarily, CDK8 variant are at least about 10 residues in length, alternatively at least about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600 in length, or more. Optionally, CDK8 variant will have or encode a sequence having no more than one conservative amino acid substitution as compared to CDK8, alternatively no more than 2, 3, 4, 5, 6, 7, 8, 9, or 10 conservative amino acid substitution as compared to CDK8.

The term “CDK8 antagonist” as defined herein is any molecule that partially or fully blocks, inhibits, or neutralizes a biological activity mediated by a native sequence CDK8. In certain embodiments such antagonist binds to CDK8. According to one embodiment, the antagonist is a polypeptide. According to another embodiment, the antagonist is an anti-CDK8 antibody. According to another embodiment, the antagonist is a small molecule antagonist. According to another embodiment, the antagonist is a polynucleotide antagonist.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Oligonucleotide,” as used herein, generally refers to short, single stranded, polynucleotides that are, but not necessarily, less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “primer” refers to a single stranded polynucleotide that is capable of hybridizing to a nucleic acid and following polymerization of a complementary nucleic acid, generally by providing a free 3′-OH group.

The term “small molecule” refers to any molecule with a molecular weight of about 2000 daltons or less, preferably of about 500 daltons or less.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The terms “anti-CDK8 antibody” and “an antibody that binds to CDK8” refer to an antibody that is capable of binding CDK8 with sufficient affinity such that the antibody is useful as a diagnostic and/or therapeutic agent in targeting CDK8. In one embodiment, the extent of binding of an anti-CDK8 antibody to an unrelated, non-CDK8 protein is less than about 10% of the binding of the antibody to CDK8 as measured, e.g., by a radioimmunoassay (RIA). In certain embodiments, an anti-CDK8 antibody binds to an epitope of CDK8 that is conserved among CDK8 from different species.

A “blocking” antibody or an “antagonist” antibody is one which inhibits or reduces biological activity of the antigen it binds. Preferred blocking antibodies or antagonist antibodies substantially or completely inhibit the biological activity of the antigen.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The terms “full length antibody,” “intact antibody,” and “whole antibody” are used herein interchangeably to refer to an antibody having a structure substantially similar to a native antibody structure or having heavy chains that contain an Fc region as defined herein.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

An “immunoconjugate” is an antibody conjugated to one or more heterologous molecule(s), including but not limited to a cytotoxic agent.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “detection” includes any means of detecting, including direct and indirect detection.

The terms “cancer stem cell-like properties” and “cancer stem cell” as used herein refers to a population of cells from a tumor that: (1) have extensive proliferative capacity; (2) are capable of asymmetric cell division to generate one or more kinds of differentiated progeny with reduced proliferative or developmental potential; (3) are capable of symmetric cell divisions for self-renewal or self-maintenance; and/or, (4) are capable of forming palpable tumors upon serial transplantation in a xenograft model. In some embodiments, the properties of enhanced proliferative capacity and asymmetric and symmetric cell division of “cancer stem cells” confer on those cancer stem cells the ability to form palpable tumors upon serial transplantation into an immuno-compromised mouse compared to the majority of tumor cells that fail to generate tumors.

The term “biomarker” as used herein refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features. In some embodiments, a biomarker is a gene. Biomarkers include, but are not limited to, polynucleotides (e.g., DNA, and/or RNA), polypeptides, polypeptide and polynucleotide modifications (e.g. posttranslational modifications), carbohydrates, and/or glycolipid-based molecular markers.

The terms “biomarker signature,” “signature,” “biomarker expression signature,” or “expression signature” are used interchangeably herein and refer to one or a combination of biomarkers whose expression is an indicator, e.g., predictive, diagnostic, and/or prognostic. The biomarker signature may serve as an indictor of a particular subtype of a disease or disorder (e.g., cancer) characterized by certain molecular, pathological, histological, and/or clinical features. In some embodiments, the biomarker signature is a “gene signature.” The term “gene signature” is used interchangeably with “gene expression signature” and refers to one or a combination of polynucleotides whose expression is an indicator, e.g., predictive, diagnostic, and/or prognostic. In some embodiments, the biomarker signature is a “protein signature.” The term “protein signature” is used interchangeably with “protein expression signature” and refers to one or a combination of polypeptides whose expression is an indicator, e.g., predictive, diagnostic, and/or prognostic.

The “amount” or “level” of a biomarker associated with an increased clinical benefit to an individual is a detectable level in a biological sample. These can be measured by methods known to one skilled in the art and also disclosed herein. The expression level or amount of biomarker assessed can be used to determine the response to the treatment.

The terms “level of expression” or “expression level” in general are used interchangeably and generally refer to the amount of a biomarker in a biological sample. “Expression” generally refers to the process by which information (e.g., gene-encoded and/or epigenetic) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

“Elevated expression,” “elevated expression levels,” or “elevated levels” refers to an increased expression or increased levels of a biomarker in an individual relative to a control, such as an individual or individuals who are not suffering from the disease or disorder (e.g., cancer) or an internal control (e.g., housekeeping biomarker).

“Reduced expression,” “reduced expression levels,” or “reduced levels” refers to a decrease expression or decreased levels of a biomarker in an individual relative to a control, such as an individual or individuals who are not suffering from the disease or disorder (e.g., cancer) or an internal control (e.g., housekeeping biomarker).

The term “housekeeping biomarker” refers to a biomarker or group of biomarkers (e.g., polynucleotides and/or polypeptides) which are typically similarly present in all cell types. In some embodiments, the housekeeping biomarker is a “housekeeping gene.” A “housekeeping gene” refers herein to a gene or group of genes which encode proteins whose activities are essential for the maintenance of cell function and which are typically similarly present in all cell types.

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The term “multiplex-PCR” refers to a single PCR reaction carried out on nucleic acid obtained from a single source (e.g., an individual) using more than one primer set for the purpose of amplifying two or more DNA sequences in a single reaction.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) overnight hybridization in a solution that employs 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with a 10 minute wash at 42° C. in 0.2×SSC (sodium chloride/sodium citrate) followed by a 10 minute high-stringency wash consisting of 0.1×SSC containing EDTA at 55° C.

“Moderately stringent conditions” can be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “diagnosis” is used herein to refer to the identification or classification of a molecular or pathological state, disease or condition (e.g., cancer). For example, “diagnosis” may refer to identification of a particular type of cancer. “Diagnosis” may also refer to the classification of a particular subtype of cancer, e.g., by histopathological criteria, or by molecular features (e.g., a subtype characterized by expression of one or a combination of biomarkers (e.g., particular genes or proteins encoded by said genes)).

The term “aiding diagnosis” is used herein to refer to methods that assist in making a clinical determination regarding the presence, or nature, of a particular type of symptom or condition of a disease or disorder (e.g., cancer). For example, a method of aiding diagnosis of a disease or condition (e.g., cancer) can comprise measuring certain biomarkers in a biological sample from an individual.

The term “sample,” as used herein, refers to a composition that is obtained or derived from a subject and/or individual of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. Samples include, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

A “reference sample”, “reference cell”, “reference tissue”, “control sample”, “control cell”, or “control tissue”, as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual. For example, healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

For the purposes herein a “section” of a tissue sample is meant a single part or piece of a tissue sample, e.g. a thin slice of tissue or cells cut from a tissue sample. It is understood that multiple sections of tissue samples may be taken and subjected to analysis, provided that it is understood that the same section of tissue sample may be analyzed at both morphological and molecular levels, or analyzed with respect to both polypeptides and polynucleotides.

By “correlate” or “correlating” is meant comparing, in any way, the performance and/or results of a first analysis or protocol with the performance and/or results of a second analysis or protocol. For example, one may use the results of a first analysis or protocol in carrying out a second protocols and/or one may use the results of a first analysis or protocol to determine whether a second analysis or protocol should be performed. With respect to the embodiment of polynucleotide analysis or protocol, one may use the results of the polynucleotide expression analysis or protocol to determine whether a specific therapeutic regimen should be performed.

“Individual response” or “response” can be assessed using any endpoint indicating a benefit to the individual, including, without limitation, (1) inhibition, to some extent, of disease progression (e.g., cancer progression), including slowing down and complete arrest; (2) a reduction in tumor size; (3) inhibition (i.e., reduction, slowing down or complete stopping) of cancer cell infiltration into adjacent peripheral organs and/or tissues; (4) inhibition (i.e. reduction, slowing down or complete stopping) of metasisis; (5) relief, to some extent, of one or more symptoms associated with the disease or disorder (e.g., cancer); (6) increase in the length of progression free survival; and/or (9) decreased mortality at a given point of time following treatment.

The term “substantially the same” or “non-differential” as used herein, denotes a sufficiently high degree of similarity between two numeric values, such that one of skill in the art would consider the difference between the two values to be of little or no biological and/or statistical significance within the context of the biological characteristic measured by said values (e.g., Kd values or expression). The difference between said two values is, for example, less than about 50%, less than about 40%, less than about 30%, less than about 20%, and/or less than about 10% as a function of the reference/comparator value.

The phrase “substantially different” or “differential” as used herein, denotes a sufficiently high degree of difference between two numeric values such that one of skill in the art would consider the difference between the two values to be of statistical significance within the context of the biological characteristic measured by said values (e.g., Kd values). The difference between said two values is, for example, greater than about 10%, greater than about 20%, greater than about 30%, greater than about 40%, and/or greater than about 50% as a function of the value for the reference/comparator molecule.

The word “label” when used herein refers to a detectable compound or composition. The label is typically conjugated or fused directly or indirectly to a reagent, such as a polynucleotide probe or an antibody, and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which results in a detectable product.

An “effective amount” of an agent refers to an amount effective, at dosages and for periods of time necessary, to achieve the desired therapeutic or prophylactic result.

A “therapeutically effective amount” of a substance/molecule of the invention, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “anti-cancer therapy” refers to a therapy useful in treating cancer. Examples of anti-cancer therapeutic agents include, but are limited to, e.g., chemotherapeutic agents, growth inhibitory agents, cytotoxic agents, agents used in radiation therapy, anti-angiogenesis agents, apoptotic agents, anti-tubulin agents, and other agents to treat cancer, anti-CD20 antibodies, platelet derived growth factor inhibitors (e.g., Gleevec™ (Imatinib Mesylate)), a COX-2 inhibitor (e.g., celecoxib), interferons, cytokines, antagonists (e.g., neutralizing antibodies) that bind to one or more of the following targets PDGFR-beta, BlyS, APRIL, BCMA receptor(s), TRAIL/Apo2, and other bioactive and organic chemical agents, etc. Combinations thereof are also included in the invention.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents e.g., methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

A “chemotherapeutic agent” refers to a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Nicolaou et al., Angew. Chem. Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®), albumin-engineered nanoparticle formulation of paclitaxel (ABRAXANE™), and docetaxel (TAXOTERE®); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g., celecoxib or etoricoxib), proteosome inhibitor (e.g., PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors (see definition below); tyrosine kinase inhibitors (see definition below); serine-threonine kinase inhibitors such as rapamycin (sirolimus, RAPAMUNE®); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents as defined herein include “anti-hormonal agents” or “endocrine therapeutics” which act to regulate, reduce, block, or inhibit the effects of hormones that can promote the growth of cancer. They may be hormones themselves, including, but not limited to: anti-estrogens with mixed agonist/antagonist profile, including, tamoxifen (NOLVADEX®), 4-hydroxytamoxifen, toremifene (FARESTON®), idoxifene, droloxifene, raloxifene (EVISTA®), trioxifene, keoxifene, and selective estrogen receptor modulators (SERMs) such as SERM3; pure anti-estrogens without agonist properties, such as fulvestrant (FASLODEX®), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (AROMASIN®), and nonsteroidal aromatase inhibitors such as anastrazole (ARIMIDEX®), letrozole (FEMARA®) and aminoglutethimide, and other aromatase inhibitors include vorozole (RIVISOR®), megestrol acetate (MEGASE®), fadrozole, and 4(5)-imidazoles; lutenizing hormone-releasing hormone agonists, including leuprolide (LUPRON® and ELIGARD®), goserelin, buserelin, and tripterelin; sex steroids, including progestines such as megestrol acetate and medroxyprogesterone acetate, estrogens such as diethylstilbestrol and premarin, and androgens/retinoids such as fluoxymesterone, all transretionic acid and fenretinide; onapristone; anti-progesterones; estrogen receptor down-regulators (ERDs); anti-androgens such as flutamide, nilutamide and bicalutamide; and pharmaceutically acceptable salts, acids or derivatives of any of the above; as well as combinations of two or more of the above.

The term “prodrug” as used in this application refers to a precursor or derivative form of a pharmaceutically active substance that is less cytotoxic to tumor cells compared to the parent drug and is capable of being enzymatically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

A “growth inhibitory agent” when used herein refers to a compound or composition which inhibits growth of a cell (e.g., a cell whose growth is dependent upon CDK8 expression either in vitro or in vivo). Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al., (WB Saunders: Philadelphia, 1995), especially p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one time administration and typical dosages range from 10 to 200 units (Grays) per day.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

By “reduce or inhibit” is meant the ability to cause an overall decrease of 20%, 30%, 40%, 50%, 60%, 70%, 75%, 80%, 85%, 90%, 95%, or greater. Reduce or inhibit can refer to the symptoms of the disorder being treated, the presence or size of metastases, or the size of the primary tumor.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a probe for specifically detecting a biomarker described herein. In certain embodiments, the manufacture or kit is promoted, distributed, or sold as a unit for performing the methods described herein.

A “target audience” is a group of people or an institution to whom or to which a particular medicament is being promoted or intended to be promoted, as by marketing or advertising, especially for particular uses, treatments, or indications, such as individuals, populations, readers of newspapers, medical literature, and magazines, television or internet viewers, radio or internet listeners, physicians, drug companies, etc.

As is understood by one skilled in the art, reference to “about” a value or parameter herein includes (and describes) embodiments that are directed to that value or parameter per se. For example, description referring to “about X” includes description of “X”.

It is understood that aspect and embodiments of the invention described herein include “consisting” and/or “consisting essentially of” aspects and embodiments. As used herein, the singular form “a”, “an”, and “the” includes plural references unless indicated otherwise.

### II. Methods and Uses

Provided herein are methods utilizing a CDK8 antagonist. For example, in some embodiments, provided herein are methods of treating a disease or disorder in an individual comprising administering to the individual an effective amount of a CDK8 antagonist, wherein treatment is based upon differential expression levels of one or more biomarkers of a CDK8 gene signature in a sample from the individual (e.g., compared to a reference sample, reference cell, reference tissue, control sample, control cell, control tissue, or internal control (e.g., housekeeping gene)). In some embodiments, differential expression of one or more biomarkers of the CDK8 gene signature is elevated expression of one or more CDK8-induced biomarkers of the CDK8 gene signature and/or reduced expression of one or more CDK8-repressed biomarkers of the CDK8 gene signature.

Also provided herein are methods of treating a cancer cell, wherein the cancer cell differentially expresses one or more biomarkers of a CDK8 gene signature (e.g., compared to a reference sample, reference cell, reference tissue, control sample, control cell, control tissue, or internal control (e.g., housekeeping gene)), the method comprising providing an effective amount of a CDK8 antagonist. In some embodiments, differential expression of one or more biomarkers of the CDK8 gene signature is elevated expression of one or more CDK8-induced biomarkers of the CDK8 gene signature and/or reduced expression of one or more CDK8-repressed biomarkers of the CDK8 gene signature.

Also provided are methods of treating a disease or disorder in an individual comprising administering to the individual an effective amount of a CDK8 antagonist, wherein treatment is continued based upon differential expression levels of one or more biomarkers of a CDK8 gene signature in a sample from the individual (e.g., compared to a reference sample, reference cell, reference tissue, control sample, control cell, control tissue, or internal control (e.g., housekeeping gene)). In some embodiments, differential expression of one or more biomarkers of the CDK8 gene signature is reduced expression of one or more CDK8-induced biomarkers of the CDK8 gene signature and/or elevated expression of one or more CDK8-repressed biomarkers of the CDK8 gene signature.

Provided herein are methods for treating a disease or disorder in an individual, the method comprising: determining that a sample obtained from the individual comprises differential expression levels of one or more biomarkers of a CDK8 gene signature (e.g., compared to a reference sample, reference cell, reference tissue, control sample, control cell, control tissue, or internal control (e.g., housekeeping gene)), and administering an effective amount of a CDK8 antagonist to the individual, whereby the disease or disorder is treated. In some embodiments, differential expression of one or more biomarkers of the CDK8 gene signature is elevated expression of one or more CDK8-induced biomarkers of the CDK8 gene signature and/or reduced expression of one or more CDK8-reduced biomarkers of the CDK8 gene signature.

Methods are also provided herein for treating disease or disorder in an individual, comprising: (a) selecting an individual having differential expression levels of one or more biomarkers of a CDK8 gene signature in a sample from the individual (e.g., compared to a reference sample, reference cell, reference tissue, control sample, control cell, control tissue, or internal control (e.g., housekeeping gene)); and (b) administering to the individual thus selected an effective amount of a CDK8 antagonist, whereby the disease or disorder is treated. In some embodiments, differential expression of one or more biomarkers of the CDK8 gene signature is elevated expression of one or more CDK8-induced biomarkers of the CDK8 gene signature and/or reduced expression of one or more CDK8-repressed biomarkers of the CDK8 gene signature.

Provided are methods of identifying an individual with a disease or disorder who is more or less likely to exhibit benefit from treatment with a therapy comprising a CDK8 antagonist, the method comprising: determining the expression levels of one or more biomarkers of a CDK8 gene signature in a sample from the individual, wherein differential expression levels of one or more biomarkers of the CDK8 gene signature in a sample from the individual (e.g., compared to a reference sample, reference cell, reference tissue, control sample, control cell, control tissue, or internal control (e.g., housekeeping gene)) indicates that the individual is more likely to exhibit benefit from treatment with the therapy comprising the CDK8 antagonist and/or non-differential expression levels of one or more biomarkers of the CDK8 gene signature in a sample from the individual (e.g., compared to a reference sample, reference cell, reference tissue, control sample, control cell, control tissue, or internal control (e.g., housekeeping gene)) indicates that the individual is less likely to exhibit benefit from treatment with the therapy comprising the CDK8 antagonist. In some embodiments, the method further comprises administering an effective amount of a therapy comprising a CDK8 antagonist. In some embodiments, differential expression of one or more biomarkers of the CDK8 gene signature is elevated expression of one or more CDK8-induced biomarkers of the CDK8 gene signature and/or reduced expression of one or more CDK8-repressed biomarkers of the CDK8 gene signature.

Further provided herein are methods for predicting whether an individual with a disease or disorder is more or less likely to respond effectively to treatment with a therapy comprising a CDK8 antagonist, the method comprising assessing expression levels of one or more biomarkers of a CDK8 gene signature in a sample from the individual, whereby differential expression levels of one or more biomarkers of the CDK8 gene signature in a sample from the individual (e.g., compared to a reference sample, reference cell, reference tissue, control sample, control cell, control tissue, or internal control (e.g., housekeeping gene)) indicates that the individual is more likely to respond effectively to treatment with the CDK8 antagonist and/or non-differential expression levels of one or more biomarkers of the CDK8 gene signature in a sample from the individual (e.g., compared to a reference sample, reference cell, reference tissue, control sample, control cell, control tissue, or internal control (e.g., housekeeping gene)) indicates that the individual is less likely to respond effectively to treatment with the CDK8 antagonist. In some embodiments, differential expression of one or more biomarkers of the CDK8 gene signature is elevated expression of one or more CDK8-induced biomarkers of the CDK8 gene signature and/or reduced expression of one or more CDK8-repressed biomarkers of the CDK8 gene signature.

Further provided are methods of predicting the response or lack of response of an individual with a disease or disorder to a therapy comprising a CDK8 antagonist comprising measuring expression levels of one or more biomarkers of a CDK8 gene signature in a sample from the individual, wherein differential expression levels of one or more biomarkers of the CDK8 gene signature in a sample from the individual (e.g., compared to a reference sample, reference cell, reference tissue, control sample, control cell, control tissue, or internal control (e.g., housekeeping gene)) is predictive of response of the individual to the therapy comprising the CDK8 antagonist and non-differential expression levels of one or more biomarkers of the CDK8 gene signature in a sample from the individual (e.g., compared to a reference sample, reference cell, reference tissue, control sample, control cell, control tissue, or internal control (e.g., housekeeping gene)) is predictive of lack of response of the individual to the therapy comprising the CDK8 antagonist. In some embodiments, the method further comprises administering an effective amount of a therapy comprising a CDK8 antagonist. In some embodiments, differential expression of one or more biomarkers of the CDK8 gene signature is elevated expression of one or more CDK8-induced biomarkers of the CDK8 gene signature and/or reduced expression of one or more CDK8-repressed biomarkers of the CDK8 gene signature.

Provided herein are methods of determining whether an individual having a disease or disorder is more or less likely responding to therapy, wherein therapy comprises a CDK8 antagonist, based upon levels of one or more biomarkers of a CDK8 gene signature in a sample from the individual, wherein differential expression levels of one or more biomarkers of the CDK8 gene signature in a sample from the individual (e.g., compared to a reference sample, reference cell, reference tissue, control sample, control cell, control tissue, or internal control (e.g., housekeeping gene)) identifies the individual as more likely responding to therapy comprising the CDK8 antagonist and non-differential expression levels of one or more biomarkers of the CDK8 gene signature in a sample from the individual (e.g., compared to a reference sample, reference cell, reference tissue, control sample, control cell, control tissue, or internal control (e.g., housekeeping gene)) identifies the individual as less likely responding to therapy comprising the CDK8 antagonist. In some embodiments, differential expression of one or more biomarkers of the CDK8 gene signature is reduced expression of one or more CDK8-repressed biomarkers of the CDK8 gene signature and/or elevated expression of one or more CDK8-induced biomarkers of the CDK8 gene signature.

Also provided herein are methods of promoting differentiation of a stem cell and/or a cancer stem cell comprising contacting the cell with an effective amount of CDK8 antagonist. Provided herein are methods of treating cancer, wherein the cancer comprises cancer stem cell-like properties comprising administering to an individual an effective amount of a therapy comprising a CDK8 antagonist. In some embodiments, the CDK8 antagonist promotes differentiation of the cancer stem cell. In some embodiments, the cancer stem cell differentiates into a goblet cell and/or enterocyte cell. In some embodiments, the CDK8 antagonist inhibits growth and/or proliferation of the cancer. In some embodiments, the cancer stem cell-like properties comprise differential expression of one or more gene of the CDK8 signature.

In some embodiments, the one or more biomarkers of the CDK8 gene signature comprises one or more biomarkers of a CDK8 cancer cell gene signature. In some embodiments, the cancer cell is a colorectal cancer cell. In some embodiments, the cancer cell is a colon cancer cell. In some embodiments, the one or more biomarkers of a CDK8 cancer cell gene signature comprises one or more biomarkers of Table 2. In some embodiments, the one or more biomarkers listed in Table 2 comprises one or more ES cell-related genes. In some embodiments, the one or more biomarkers listed in Table 2 comprises one or more MYC ES target genes. In some embodiments, the one or more biomarkers listed in Table 2 comprises one or more p53 signalling genes, cell cycle genes, Wnt signalling genes, and/or SMAD/BMP signalling genes. In some embodiments, the one or more biomarkers listed in Table 2 does not comprise (e.g., excludes) ES genes and/or MYC ES target genes. In some embodiments, the one or more biomarkers listed in Table 2 comprises one or more p53 signalling genes, cell cycle genes, Wnt signalling genes, and/or SMAD/BMP signalling genes, but is not a MYC ES target gene and/or ES genes. In some embodiments, the one or more biomarkers of the CDK8 gene signature comprises one or more biomarkers of a CDK8 embryonic stem cell gene signature. In some embodiments, the one or more biomarkers of a CDK8 embryonic stem cell gene signature comprises one or more biomarkers of Table 3. In some embodiments, the one or more biomarkers of the CDK8 gene signature comprises one or more genes selected from the group consisting of SABP5, LEAP2, SKP2, CDK6, DICER1, LYAR, RNF138, STIL, POLD3, JAG2, OBRC2A, PPARGCIB, TPD52L2, MRPL12, NUCKS1, and GEMIN5.

In some embodiments of any of the methods, the one or more biomarkers of the CDK8 gene signature in Tables 2 and/or 3 have a P-value of greater than about any of 1×10⁻², 1×10⁻³, 1×10⁻⁴, 1×10⁻⁵, 1×10⁻⁶, 1×10⁻⁷, 1×10⁻⁸, 1×10⁻⁹, and/or 1×10⁻¹⁰.

In some embodiments of any of the methods, the one or more biomarkers of the CDK8 gene signature, one or more biomarkers of a CDK8 cancer cell gene signature, and/or one or more biomarkers of a CDK8 embryonic stem cell gene signature includes greater than about any of 5, 10, 25, 50, 100, 175, 250, 375, 500, 625, 750, 875, 1000, 1125, 1250, 1375 and/or 1500 biomarkers listed in Table 2 and/or 3. In some embodiments of any of the methods, the one or more biomarkers of the CDK8 gene signature, one or more biomarkers of a CDK8 cancer cell gene signature, and/or one or more biomarkers of a CDK8 embryonic stem cell gene signature includes all of the biomarkers listed in Table 2 and/or 3. In some embodiments of any of the methods, the one or more biomarkers of the CDK8 gene signature includes all of the biomarkers listed in Table 2 and 3.

In some embodiments of any of the methods, the disease or disorder is an angiogenesis disease or disorder, proliferative disease or disorder, and/or an angiogenic disease or disorder. In some embodiments, the disease or disorder is a tumor and/or cancer. Examples of cancers and cancer cells include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g., epithelial squamous cell cancer), lung cancer including small-cell lung cancer (SCLC), non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophageal cancer, tumors of the biliary tract, as well as head and neck cancer. In some embodiments, the cancer is metastatic cancer. In some embodiments, the cancer is colorectal cancer. In some embodiments, the cancer is colon cancer.

In some embodiments of any of the methods, differential expression levels of one or more biomarkers of a CDK8 gene signature is elevated expression. In some embodiments, elevated expression refers to an overall increase of about any of 10%, 20%, 25%, 30%, 40%, 50%, 60%, 70%, 80%, 85%, 90%, 95%, 96%, 97%, 98%, 99% or greater, in the level of biomarker (e.g., protein or nucleic acid (e.g., gene or mRNA)), detected by standard art known methods such as those described herein, as compared to a reference sample, reference cell, reference tissue, control sample, control cell, or control tissue. In certain embodiments, the elevated expression refers to the increase in expression level/amount of a biomarker in the sample wherein the increase is at least about any of 1.5×, 1.75×, 2×, 3×, 4×, 5×, 6×, 7×, 8×, 9×, 10×, 25×, 50×, 75×, or 100× the expression level/amount of the respective biomarker in a reference sample, reference cell, reference tissue, control sample, control cell, or control tissue. In some embodiments, elevated expression refers to an overall increase of greater than about any of 1.05 fold, 1.1 fold, 1.2 fold, 1.3 fold, 1.4 fold, 1.5 fold, about 1.75 fold, about 2 fold, about 2.25 fold, about 2.5 fold, about 2.75 fold, about 3.0 fold, or about 3.25 fold as compared to a reference sample, reference cell, reference tissue, control sample, control cell, control tissue, or internal control (e.g., housekeeping gene).

In some embodiments of any of the methods, differential expression levels of one or more biomarkers of a CDK8 gene signature is reduced expression. In some embodiments, reduced expression refers to an overall reduction of about any of 10%, 20%, 25%, 30%, 40%, 50%, 60%, 70%, 80%, 85%, 90%, 95%, 96%, 97%, 98%, 99% or greater, in the level of biomarker (e.g., protein or nucleic acid (e.g., gene or mRNA)), detected by standard art known methods such as those described herein, as compared to a reference sample, reference cell, reference tissue, control sample, control cell, or control tissue. In certain embodiments, reduced expression refers to the decrease in expression level/amount of a biomarker in the sample wherein the decrease is at least about any of 0.9×, 0.8×, 0.7×, 0.6×, 0.5×, 0.4×, 0.3×, 0.2×, 0.1×, 0.05×, or 0.01× the expression level/amount of the respective biomarker in a reference sample, reference cell, reference tissue, control sample, control cell, or control tissue.

Presence and/or expression levels/amount of a biomarker of the CDK8 gene signature can be determined qualitatively and/or quantitatively based on any suitable criterion known in the art, including but not limited to DNA, mRNA, cDNA, proteins, protein fragments and/or gene copy number. In certain embodiments, presence and/or expression levels/amount of a biomarker in a first sample is increased as compared to presence/absence and/or expression levels/amount in a second sample. In certain embodiments, presence/absence and/or expression levels/amount of a biomarker in a first sample is decreased as compared to presence and/or expression levels/amount in a second sample. In certain embodiments, the second sample is a reference sample, reference cell, reference tissue, control sample, control cell, or control tissue. Additional disclosures for determining presence/absence and/or expression levels/amount of a gene are described herein. In some embodiments, the reference gene is CD133 and/or CD44.

Presence and/or expression level/amount of various biomarkers in a sample can be analyzed by a number of methodologies, many of which are known in the art and understood by the skilled artisan, including, but not limited to, immunohistochemical (“IHC”), Western blot analysis, immunoprecipitation, molecular binding assays, ELISA, ELIFA, fluorescence activated cell sorting (“FACS”), MassARRAY, proteomics, quantitative blood based assays (as for example Serum ELISA), biochemical enzymatic activity assays, in situ hybridization, Southern analysis, Northern analysis, whole genome sequencing, polymerase chain reaction (“PCR”) including quantitative real time PCR (“qRT-PCR”) and other amplification type detection methods, such as, for example, branched DNA, SISBA, TMA and the like), RNA-Seq, FISH, microarray analysis, gene expression profiling, and/or serial analysis of gene expression (“SAGE”), as well as any one of the wide variety of assays that can be performed by protein, gene, and/or tissue array analysis. Typical protocols for evaluating the status of genes and gene products are found, for example in Ausubel et al., eds., 1995, Current Protocols In Molecular Biology, Units 2 (Northern Blotting), 4 (Southern Blotting), (Immunoblotting) and 18 (PCR Analysis). Multiplexed immunoassays such as those available from Rules Based Medicine or Meso Scale Discovery (“MSD”) may also be used.

In some embodiments, presence and/or expression level/amount of a biomarker is determined using a method comprising: (a) performing gene expression profiling, PCR (such as rtPCR), RNA-seq, microarray analysis, SAGE, MassARRAY technique, or FISH on a sample (such as an subject cancer sample); and b) determining presence and/or expression level/amount of a biomarker in the sample. In some embodiments, the microarray method comprises the use of a microarray chip having one or more nucleic acid molecules that can hybridize under stringent conditions to a nucleic acid molecule encoding a gene mentioned above or having one or more polypeptides (such as peptides or antibodies) that can bind to one or more of the proteins encoded by the genes mentioned above. In one embodiment, the PCR method is qRT-PCR. In one embodiment, the PCR method is multiplex-PCR. In some embodiments, gene expression is measured by microarray. In some embodiments, gene expression is measured by qRT-PCR. In some embodiments, expression is measured by multiplex-PCR.

Methods for the evaluation of mRNAs in cells are well known and include, for example, hybridization assays using complementary DNA probes (such as in situ hybridization using labeled riboprobes specific for the one or more genes, Northern blot and related techniques) and various nucleic acid amplification assays (such as RT-PCR using complementary primers specific for one or more of the genes, and other amplification type detection methods, such as, for example, branched DNA, SISBA, TMA and the like).

Samples from mammals can be conveniently assayed for mRNAs using Northern, dot blot or PCR analysis. In addition, such methods can include one or more steps that allow one to determine the levels of target mRNA in a biological sample (e.g., by simultaneously examining the levels a comparative control mRNA sequence of a “housekeeping” gene such as an actin family member). Optionally, the sequence of the amplified target cDNA can be determined.

Optional methods of the invention include protocols which examine or detect mRNAs, such as target mRNAs, in a tissue or cell sample by microarray technologies. Using nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes whose expression correlates with increased or reduced clinical benefit of anti-angiogenic therapy may be arrayed on a solid support. Hybridization of a labeled probe with a particular array member indicates that the sample from which the probe was derived expresses that gene.

According to some embodiments, presence and/or expression level/amount is measured by observing protein expression levels of an aforementioned gene. In certain embodiments, the method comprises contacting the biological sample with antibodies to a biomarker described herein under conditions permissive for binding of the biomarker, and detecting whether a complex is formed between the antibodies and biomarker. Such method may be an in vitro or in vivo method. In one embodiment, an antibody is used to select subjects eligible for therapy with CDK8 antagonist, e.g., a biomarker for selection of individuals.

In certain embodiments, the presence and/or expression level/amount of biomarker proteins in a sample is examined using IHC and staining protocols. IHC staining of tissue sections has been shown to be a reliable method of determining or detecting presence of proteins in a sample. In one aspect, expression level of biomarker is determined using a method comprising: (a) performing IHC analysis of a sample (such as a subject cancer sample) with an antibody; and b) determining expression level of a biomarker in the sample. In some embodiments, 1HC staining intensity is determined relative to a reference value.

IHC may be performed in combination with additional techniques such as morphological staining and/or fluorescence in-situ hybridization. Two general methods of IHC are available; direct and indirect assays. According to the first assay, binding of antibody to the target antigen is determined directly. This direct assay uses a labeled reagent, such as a fluorescent tag or an enzyme-labeled primary antibody, which can be visualized without further antibody interaction. In a typical indirect assay, unconjugated primary antibody binds to the antigen and then a labeled secondary antibody binds to the primary antibody. Where the secondary antibody is conjugated to an enzymatic label, a chromogenic or fluorogenic substrate is added to provide visualization of the antigen. Signal amplification occurs because several secondary antibodies may react with different epitopes on the primary antibody.

The primary and/or secondary antibody used for IHC typically will be labeled with a detectable moiety. Numerous labels are available which can be generally grouped into the following categories: (a) Radioisotopes, such as 35S, 14C, 125I, 3H, and 131I; (b) colloidal gold particles; (c) fluorescent labels including, but are not limited to, rare earth chelates (europium chelates), Texas Red, rhodamine, fluorescein, dansyl, Lissamine, umbelliferone, phycocrytherin, phycocyanin, or commercially available fluorophores such SPECTRUM ORANGE7 and SPECTRUM GREEN7 and/or derivatives of any one or more of the above; (d) various enzyme-substrate labels are available and U.S. Pat. No. 4,275,149 provides a review of some of these. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like.

Examples of enzyme-substrate combinations include, for example, horseradish peroxidase (HRPO) with hydrogen peroxidase as a substrate; alkaline phosphatase (AP) with para-Nitrophenyl phosphate as chromogenic substrate; and β-D-galactosidase (β-D-Gal) with a chromogenic substrate (e.g., p-nitrophenyl-β-D-galactosidase) or fluorogenic substrate (e.g., 4-methylumbelliferyl-β-D-galactosidase). For a general review of these, see U.S. Pat. Nos. 4,275,149 and 4,318,980.

Specimens thus prepared may be mounted and coverslipped. Slide evaluation is then determined, e.g., using a microscope, and staining intensity criteria, routinely used in the art, may be employed. In some embodiments, a staining pattern score of about 1+ or higher is diagnostic and/or prognostic. In certain embodiments, a staining pattern score of about 2+ or higher in an IHC assay is diagnostic and/or prognostic. In other embodiments, a staining pattern score of about 3 or higher is diagnostic and/or prognostic. In one embodiment, it is understood that when cells and/or tissue from a tumor or colon adenoma are examined using IHC, staining is generally determined or assessed in tumor cell and/or tissue (as opposed to stromal or surrounding tissue that may be present in the sample).

In alternative methods, the sample may be contacted with an antibody specific for said biomarker under conditions sufficient for an antibody-biomarker complex to form, and then detecting said complex. The presence of the biomarker may be detected in a number of ways, such as by Western blotting and ELISA procedures for assaying a wide variety of tissues and samples, including plasma or serum. A wide range of immunoassay techniques using such an assay format are available, see, e.g., U.S. Pat. Nos. 4,016,043, 4,424,279 and 4,018,653. These include both single-site and two-site or “sandwich” assays of the non-competitive types, as well as in the traditional competitive binding assays. These assays also include direct binding of a labeled antibody to a target biomarker.

Presence and/or expression level/amount of a selected biomarker in a tissue or cell sample may also be examined by way of functional or activity-based assays. For instance, if the biomarker is an enzyme, one may conduct assays known in the art to determine or detect the presence of the given enzymatic activity in the tissue or cell sample.

In certain embodiments, the samples are normalized for both differences in the amount of the biomarker assayed and variability in the quality of the samples used, and variability between assay runs. Such normalization may be accomplished by detecting and incorporating the expression of certain normalizing biomarkers, including well known housekeeping genes, such as ACTB. Alternatively, normalization can be based on the mean or median signal of all of the assayed genes or a large subset thereof (global normalization approach). On a gene-by-gene basis, measured normalized amount of a subject tumor mRNA or protein is compared to the amount found in a reference set. Normalized expression levels for each mRNA or protein per tested tumor per subject can be expressed as a percentage of the expression level measured in the reference set. The presence and/or expression level/amount measured in a particular subject sample to be analyzed will fall at some percentile within this range, which can be determined by methods well known in the art.

In certain embodiments, relative expression level of a gene is determined as follows:

Relative expression gene1 sample1=2exp(Ct housekeeping gene−Ct gene1) with Ct determined in a sample.

Relative expression gene1 reference RNA=2exp(Ct housekeeping gene−Ct gene1) with Ct determined in the reference sample.

Normalized relative expression gene1 sample1=(relative expression gene1 sample1/relative expression gene1 reference RNA)×100

Ct is the threshold cycle. The Ct is the cycle number at which the fluorescence generated within a reaction crosses the threshold line.

All experiments are normalized to a reference RNA, which is a comprehensive mix of RNA from various tissue sources (e.g., reference RNA #636538 from Clontech, Mountain View, Calif.). Identical reference RNA is included in each qRT-PCR run, allowing comparison of results between different experimental runs.

In one embodiment, the sample is a clinical sample. In another embodiment, the sample is used in a diagnostic assay. In some embodiments, the sample is obtained from a primary or metastatic tumor. Tissue biopsy is often used to obtain a representative piece of tumor tissue. Alternatively, tumor cells can be obtained indirectly in the form of tissues or fluids that are known or thought to contain the tumor cells of interest. For instance, samples of lung cancer lesions may be obtained by resection, bronchoscopy, fine needle aspiration, bronchial brushings, or from sputum, pleural fluid or blood. Genes or gene products can be detected from cancer or tumor tissue or from other body samples such as urine, sputum, serum or plasma. The same techniques discussed above for detection of target genes or gene products in cancerous samples can be applied to other body samples. Cancer cells may be sloughed off from cancer lesions and appear in such body samples. By screening such body samples, a simple early diagnosis can be achieved for these cancers. In addition, the progress of therapy can be monitored more easily by testing such body samples for target genes or gene products.

In certain embodiments, a reference sample, reference cell, reference tissue, control sample, control cell, or control tissue is a single sample or combined multiple samples from the same subject or individual that are obtained at one or more different time points than when the test sample is obtained. For example, a reference sample, reference cell, reference tissue, control sample, control cell, or control tissue is obtained at an earlier time point from the same subject or individual than when the test sample is obtained. Such reference sample, reference cell, reference tissue, control sample, control cell, or control tissue may be useful if the reference sample is obtained during initial diagnosis of cancer and the test sample is later obtained when the cancer becomes metastatic.

In certain embodiments, a reference sample, reference cell, reference tissue, control sample, control cell, or control tissue is a combined multiple samples from one or more healthy individuals who are not the subject or individual. In certain embodiments, a reference sample, reference cell, reference tissue, control sample, control cell, or control tissue is a combined multiple samples from one or more individuals with a disease or disorder (e.g., cancer) who are not the subject or individual. In certain embodiments, a reference sample, reference cell, reference tissue, control sample, control cell, or control tissue is pooled RNA samples from normal tissues or pooled plasma or serum samples from one or more individuals who are not the subject or individual. In certain embodiments, a reference sample, reference cell, reference tissue, control sample, control cell, or control tissue is pooled RNA samples from tumor tissues or pooled plasma or serum samples from one or more individuals with a disease or disorder (e.g., cancer) who are not the subject or individual.

In some embodiments of any of the methods, the CDK8 antagonist is an antibody, binding polypeptide, small molecule, or polynucleotide. In some embodiments, the CDK8 antagonist is an antibody. In some embodiments, the antibody is a monoclonal antibody. In some embodiments, the antibody is a human, humanized, or chimeric antibody. In some embodiments, the antibody is an antibody fragment and the antibody fragment binds CDK8. In some embodiments, the CDK8 antagonist is a small molecule. In some embodiments, the small molecule is a small molecule kinase inhibitor. In some embodiments, the small molecule kinase inhibitor is selected from the group consisting of flavopiridol, ABT-869, AST-487, BMS-387032/SNS032, BIRB-796, sorafenib, staurosporine, cortistatin, cortistatin A, and/or a steroidal alkaloid or derivative thereof. In some embodiments, the CDK8 antagonist induces cell cycle arrest or is capable of promoting differentiation. In some embodiments, the CDK8 antagonist is capable of promoting a change in cell fate and promoting differentiation is indicated by reduced expression of one or more CDK8-repressed biomarkers of the CDK8 gene signature and/or elevated expression of one or more CDK8-induced biomarkers of the CDK8 gene signature.

In some embodiments of any of the methods, the individual according to any of the above embodiments may be a human.

In some embodiments of any of the methods, the method comprises administering to an individual having such cancer an effective amount of a CDK8 antagonist. In one such embodiment, the method further comprises administering to the individual an effective amount of at least one additional therapeutic agent, as described below. In some embodiments, the individual may be a human.

The CDK8 antagonist described herein can be used either alone or in combination with other agents in a therapy. For instance, a CDK8 antagonist, described herein may be co-administered with at least one additional therapeutic agent including another CDK8 antagonist. In certain embodiments, an additional therapeutic agent is a chemotherapeutic agent.

Such combination therapies noted above encompass combined administration (where two or more therapeutic agents are included in the same or separate formulations), and separate administration, in which case, administration of the CDK8 antagonist can occur prior to, simultaneously, and/or following, administration of the additional therapeutic agent and/or adjuvant. CDK8 antagonist can also be used in combination with radiation therapy.

A CDK8 antagonist (e.g., an antibody, binding polypeptide, and/or small molecule) described herein (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

CDK8 antagonist (e.g., an antibody, binding polypeptide, and/or small molecule) described herein may be formulated, dosed, and administered in a fashion consistent with good medical practice. Factors for consideration in this context include the particular disorder being treated, the particular mammal being treated, the clinical condition of the individual, the cause of the disorder, the site of delivery of the agent, the method of administration, the scheduling of administration, and other factors known to medical practitioners. The CDK8 antagonist, need not be, but is optionally formulated with one or more agents currently used to prevent or treat the disorder in question. The effective amount of such other agents depends on the amount of the CDK8 antagonist, present in the formulation, the type of disorder or treatment, and other factors discussed above. These are generally used in the same dosages and with administration routes as described herein, or about from Ito 99% of the dosages described herein, or in any dosage and by any route that is empirically/clinically determined to be appropriate.

For the prevention or treatment of disease, the appropriate dosage of a CDK8 antagonist, described herein (when used alone or in combination with one or more other additional therapeutic agents) will depend on the type of disease to be treated, the severity and course of the disease, whether the CDK8 antagonist, is administered for preventive or therapeutic purposes, previous therapy, the subject's clinical history and response to the CDK8 antagonist, and the discretion of the attending physician. The CDK8 antagonist is suitably administered to the individual at one time or over a series of treatments. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. Such doses may be administered intermittently, e.g., every week or every three weeks (e.g., such that the individual receives from about two to about twenty, or e.g., about six doses of the CDK8 antagonist). An initial higher loading dose, followed by one or more lower doses may be administered. An exemplary dosing regimen comprises administering. However, other dosage regimens may be useful. The progress of this therapy is easily monitored by conventional techniques and assays.

It is understood that any of the above formulations or therapeutic methods may be carried out using an immunoconjugate of the invention in place of or in addition to the CDK8 antagonist.

### III. Therapeutic Compositions

Provided herein are CDK8 antagonists useful in the methods described herein. In some embodiments, the CDK8 antagonists are an antibody, binding polypeptide, small molecule, and/or polynucleotide.

### A. Antibodies

In one aspect, provided herein isolated antibodies that bind to CDK8. In any of the above embodiments, an antibody is humanized. In a further aspect of the invention, an anti-CDK8 antibody according to any of the above embodiments is a monoclonal antibody, including a chimeric, humanized or human antibody. In one embodiment, an anti-CDK8 antibody is an antibody fragment, e.g., a Fv, Fab, Fab′, scFv, diabody, or F(ab′)₂ fragment. In another embodiment, the antibody is a full length antibody, e.g., an intact IgG1” antibody or other antibody class or isotype as defined herein.

In a further aspect, an anti-CDK8 antibody according to any of the above embodiments may incorporate any of the features, singly or in combination, as described in Sections below:

1. Antibody Affinity

In certain embodiments, an antibody provided herein has a dissociation constant (Kd) of <1 μM. In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 μM or 26 μM [¹²⁵1]-antigen antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μL1/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, N.J.) at 25° C. with immobilized antigen CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (−0.2 μM) before injection at a flow rate of 5 μL1/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μL1/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al., Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g., E. coli or phage), as described herein.

3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al., J. Immunol. 151:2296 (1993)); framework regions derived from the consensus sequence of human antibodies of a particular subgroup of light or heavy chain variable regions (see, e.g., Carter et al., Proc. Natl. Acad. Sci. USA, 89:4285 (1992); and Presta et al., J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HuMab® technology; U.S. Pat. No. 7,041,870 describing K-M MOUSE® technology, and U.S. Patent Application Publication No. US 2007/0061900, describing VelociMouse® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clin. Pharma., 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al., in METHODS IN MOL. BIOL. 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in METHODS IN MOL. BIOL. 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

6. Multispecific Antibodies

In certain embodiments, an antibody provided herein is a multispecific antibody, e.g., a bispecific antibody. Multispecific antibodies are monoclonal antibodies that have binding specificities for at least two different sites. In certain embodiments, one of the binding specificities is for CDK8 polypeptide and the other is for any other antigen. In certain embodiments, bispecific antibodies may bind to two different epitopes of CDK8 polypeptide. Bispecific antibodies may also be used to localize cytotoxic agents to cells which express CDK8 polypeptide. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al., J. Immunol. 147: 60 (1991).

Engineered antibodies with three or more functional antigen binding sites, including “Octopus antibodies,” are also included herein (see, e.g., US 2006/0025576).

The antibody or fragment herein also includes a “Dual Acting FAb” or “DAF” comprising an antigen binding site that binds to a CDK8 polypeptide as well as another, different antigen (see, US 2008/0069820, for example).

7. Antibody Variants

a) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al., TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al., J. Mol. Biol. 336:1239-1249 (2004); Yamane-Ohnuki et al., Biotech. Bioeng. 87: 614 (2004). Examples of cell lines capable of producing defucosylated antibodies include Lec13 CHO cells deficient in protein fucosylation (Ripka et al., Arch. Biochem. Biophys. 249:533-545 (1986); US 2003/0157108, Presta, L; and WO 2004/056312, Adams et al., especially at Example 11), and knockout cell lines, such as alpha-1,6-fucosyltransferase gene, FUT8, knockout CHO cells (see, e.g., Yamane-Ohnuki et al., Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

b) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al., Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al., Proc. Natl. Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).) In certain embodiments, an antibody variant comprises an Fc region with one or more amino acid substitutions which improve ADCC, e.g., substitutions at positions 298, 333, and/or 334 of the Fc region (EU numbering of residues). In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al., J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. No. 5,648,260; U.S. Pat. No. 5,624,821; and WO 94/29351 concerning other examples of Fc region variants.

c) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and 5400 (EU numbering) of the heavy chain Fc region. Cysteine engineered antibodies may be generated as described, e.g., in U.S. Pat. No. 7,521,541.

### B. Immunoconjugates

Further provided herein are immunoconjugates comprising an anti-CDK8 antibody conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example Tc⁹⁹ or I¹²³, or a spin label for nuclear magnetic resonance (NMR) imaging (also known as magnetic resonance imaging, MRI), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio)propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., USA).

### C. Binding Polypeptides

Binding polypeptides are polypeptides that bind, preferably specifically, to CDK8 as described herein. In some embodiments, the binding polypeptides are CDK8 antagonists.

Binding polypeptides may be chemically synthesized using known polypeptide synthesis methodology or may be prepared and purified using recombinant technology. Binding polypeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more, wherein such binding polypeptides that are capable of binding, preferably specifically, to a target, CDK8, as described herein. Binding polypeptides may be identified without undue experimentation using well known techniques. In this regard, it is noted that techniques for screening polypeptide libraries for binding polypeptides that are capable of specifically binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. E. et al., (1990) Proc. Natl. Acad. Sci. USA, 87:6378; Lowman, H. B. et al., (1991) Biochemistry, 30:10832; Clackson, T. et al., (1991) Nature, 352: 624; Marks, J. D. et al., (1991), J. Mol. Biol., 222:581; Kang, A. S. et al., (1991) Proc. Natl. Acad. Sci. USA, 88:8363, and Smith, G. P. (1991) Current Opin. Biotechnol., 2:668).

In this regard, bacteriophage (phage) display is one well known technique which allows one to screen large polypeptide libraries to identify member(s) of those libraries which are capable of specifically binding to a target polypeptide, CDK8 polypeptide. Phage display is a technique by which variant polypeptides are displayed as fusion proteins to the coat protein on the surface of bacteriophage particles (Scott, J. K. and Smith, G. P. (1990) Science, 249: 386). The utility of phage display lies in the fact that large libraries of selectively randomized protein variants (or randomly cloned cDNAs) can be rapidly and efficiently sorted for those sequences that bind to a target molecule with high affinity. Display of peptide (Cwirla, S. E. et al., (1990) Proc. Natl. Acad. Sci. USA, 87:6378) or protein (Lowman, H. B. et al., (1991) Biochemistry, 30:10832; Clackson, T. et al., (1991) Nature, 352: 624; Marks, J. D. et al., (1991), J. Mol. Biol., 222:581; Kang, A. S. et al., (1991) Proc. Natl. Acad. Sci. USA, 88:8363) libraries on phage have been used for screening millions of polypeptides or oligopeptides for ones with specific binding properties (Smith, G. P. (1991) Current Opin. Biotechnol., 2:668). Sorting phage libraries of random mutants requires a strategy for constructing and propagating a large number of variants, a procedure for affinity purification using the target receptor, and a means of evaluating the results of binding enrichments. U.S. Pat. Nos. 5,223,409, 5,403,484, 5,571,689, and 5,663,143.

Although most phage display methods have used filamentous phage, lambdoid phage display systems (WO 95/34683; U.S. Pat. No. 5,627,024), T4 phage display systems (Ren et al., Gene, 215: 439 (1998); Zhu et al., Cancer Research, 58(15): 3209-3214 (1998); Jiang et al., Infection & Immunity, 65(11): 4770-4777 (1997); Ren et al., Gene, 195(2):303-311 (1997); Ren, Protein Sci., 5: 1833 (1996); Efimov et al., Virus Genes, 10: 173 (1995)) and T7 phage display systems (Smith and Scott, Methods in Enzymology, 217: 228-257 (1993); U.S. Pat. No. 5,766,905) are also known.

Additional improvements enhance the ability of display systems to screen peptide libraries for binding to selected target molecules and to display functional proteins with the potential of screening these proteins for desired properties. Combinatorial reaction devices for phage display reactions have been developed (WO 98/14277) and phage display libraries have been used to analyze and control bimolecular interactions (WO 98/20169; WO 98/20159) and properties of constrained helical peptides (WO 98/20036). WO 97/35196 describes a method of isolating an affinity ligand in which a phage display library is contacted with one solution in which the ligand will bind to a target molecule and a second solution in which the affinity ligand will not bind to the target molecule, to selectively isolate binding ligands. WO 97/46251 describes a method of biopanning a random phage display library with an affinity purified antibody and then isolating binding phage, followed by a micropanning process using microplate wells to isolate high affinity binding phage. The use of Staphylococcus aureus protein A as an affinity tag has also been reported (Li et al., (1998) Mol. Biotech., 9:187). WO 97/47314 describes the use of substrate subtraction libraries to distinguish enzyme specificities using a combinatorial library which may be a phage display library. A method for selecting enzymes suitable for use in detergents using phage display is described in WO 97/09446. Additional methods of selecting specific binding proteins are described in U.S. Pat. Nos. 5,498,538, 5,432,018, and WO 98/15833.

Methods of generating peptide libraries and screening these libraries are also disclosed in U.S. Pat. Nos. 5,723,286, 5,432,018, 5,580,717, 5,427,908, 5,498,530, 5,770,434, 5,734,018, 5,698,426, 5,763,192, and 5,723,323.

### D. Small Molecules

Provided herein are small molecules for use as a CDK8 small molecule antagonist. In some embodiments, the CDK8 small molecule antagonist is flavopiridol or derivative thereof. In some embodiments, the CDK8 small molecule antagonist is ABT-869 or derivative thereof. In some embodiments, the CDK8 small molecule antagonist is AST-487 or derivative thereof. In some embodiments, the CDK8 small molecule BMS-387032/SNS032 or derivative thereof. In some embodiments, the CDK8 small molecule antagonist is BIRB-796 or derivative thereof. In some embodiments, the CDK8 small molecule antagonist is CP-724714 or derivative thereof. In some embodiments, the CDK8 small molecule antagonist is sorafenib or derivative thereof. In some embodiments, the CDK8 small molecule antagonist is staurosporine or derivative thereof. In some embodiments, the CDK8 small molecule antagonist is cortistatin or derivative thereof. In some embodiments, the CDK8 small molecule antagonist is cortistatin A or derivative thereof. In some embodiments, the CDK8 small molecule antagonist is a steroidal alkaloid or derivative thereof. In some embodiments, the CDK8 small molecule antagonist is a small molecule kinase inhibitor disclosed in Karman M. W. et al., Nature Biotech. 26(1):127-132 (2008), Schneider E. V. et al., J. Mol. Biol. 412:251-266 (2011), Cee V. J. et al., Angew. Chem. Int. Ed. 48:8952-8957 (2009), which are incorporated by reference in their entireties. Methods of screening for CDK8 small molecule antagonists are known in the art and described in Karman M. W. et al., Nature Biotech. 26(1):127-132 (2008), Schneider E. V. et al., J. Mol. Biol. 412:251-266 (2011), Cee V. J. et al., Angew. Chem. Int. Ed. 48:8952-8957 (2009), which are incorporated by reference in their entireties.

Small molecules are preferably organic molecules other than binding polypeptides or antibodies as defined herein that bind, preferably specifically, to CDK8 polypeptide as described herein. Organic small molecules may be identified and chemically synthesized using known methodology (see, e.g., PCT Publication Nos. WO00/00823 and WO00/39585). Organic small molecules are usually less than about 2000 Daltons in size, alternatively less than about 1500, 750, 500, 250 or 200 Daltons in size, wherein such organic small molecules that are capable of binding, preferably specifically, to a polypeptide as described herein may be identified without undue experimentation using well known techniques. In this regard, it is noted that techniques for screening organic small molecule libraries for molecules that are capable of binding to a polypeptide target are well known in the art (see, e.g., PCT Publication Nos. WO00/00823 and WO00/39585). Organic small molecules may be, for example, aldehydes, ketones, oximes, hydrazones, semicarbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, isocyanates, sulfonyl chlorides, diazo compounds, acid chlorides, or the like.

### E. Antagonist Polynucleotides

Provided are polynucleotide CDK8 antagonists for use in any of the methods described herein. In some embodiments, the polynucleotide CDK8 antagonists is AGCCAAGAGGAAAGAUGGG (SEQ ID NO:1), GCGAAUUACUCAGAACAG (SEQ ID NO:2), AGGUGUUUCUGUCUCAUGC (SEQ ID NO:3), UAGAAGGAACUGGGAUCUC (SEQ ID NO:4), GAATGGTGAAGTCACTATTAT (SEQ ID NO:5), CCCGATTATTTAATTCACCTT (SEQ ID NO:7), CAGGGATTTGAAACCTGCTAA (SEQ ID NO:8); shNanog, GCCAGTGATTTGGAGGTGAAT (SEQ ID NO:9), CAAAACTAGTAATCCTTATTT (SEQ ID NO:12), CCCTTACCCAAAACGAGAATT (SEQ ID NO:13), CCCATCTTTCCTCTTGGCTT (SEQ ID NO:14), CTGTTCTGAGGTAATTCGCT (SEQ ID NO:15), GCATGAGACAGAAACACCCTT (SEQ ID NO:16), GAGATCCCAGTTCCTTCTAT (SEQ ID NO:17), and/or GUUUUUFCCGGUUGUCAAA (SEQ ID NO:18). In some embodiments, the polynucleotide CDK8 antagonist is a polynucleotide CDK8 antagonist disclosed in US 2004/0180848.

The polynucleotide may be an antisense nucleic acid and/or a ribozyme. The antisense nucleic acids comprise a sequence complementary to at least a portion of an RNA transcript of a CDK8 gene. However, absolute complementarity, although preferred, is not required.

A sequence “complementary to at least a portion of an RNA,” referred to herein, means a sequence having sufficient complementarity to be able to hybridize with the RNA, forming a stable duplex; in the case of double stranded CDK8 antisense nucleic acids, a single strand of the duplex DNA may thus be tested, or triplex formation may be assayed. The ability to hybridize will depend on both the degree of complementarity and the length of the antisense nucleic acid. Generally, the larger the hybridizing nucleic acid, the more base mismatches with a CDK8 RNA it may contain and still form a stable duplex (or triplex as the case may be). One skilled in the art can ascertain a tolerable degree of mismatch by use of standard procedures to determine the melting point of the hybridized complex.

Polynucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. See generally, Wagner, R., 1994, Nature 372:333-335. Thus, oligonucleotides complementary to either the 5′- or 3′-non-translated, non-coding regions of the CDK8 gene, could be used in an antisense approach to inhibit translation of endogenous CDK8 mRNA. Polynucleotides complementary to the 5′ untranslated region of the mRNA should include the complement of the AUG start codon. Antisense polynucleotides complementary to mRNA coding regions are less efficient inhibitors of translation but could be used in accordance with the invention. Whether designed to hybridize to the 5′-, 3′- or coding region of CDK8 mRNA, antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

In one embodiment, the CDK8 antisense nucleic acid of the invention is produced intracellularly by transcription from an exogenous sequence. For example, a vector or a portion thereof, is transcribed, producing an antisense nucleic acid (RNA) of the CDK8 gene. Such a vector would contain a sequence encoding the CDK8 antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others know in the art, used for replication and expression in vertebrate cells. Expression of the sequence encoding CDK8, or fragments thereof, can be by any promoter known in the art to act in vertebrate, preferably human cells. Such promoters can be inducible or constitutive. Such promoters include, but are not limited to, the SV40 early promoter region (Bernoist and Chambon, Nature 29:304-310 (1981), the promoter contained in the 3′ long terminal repeat of Rous sarcoma virus (Yamamoto et al., Cell 22:787-797 (1980), the herpes thymidine promoter (Wagner et al., Proc. Natl. Acad. Sci. U.S.A. 78:1441-1445 (1981), the regulatory sequences of the metallothionein gene (Brinster et al., Nature 296:39-42 (1982)), etc.

### F. Antibody and Binding Polypeptide Variants

In certain embodiments, amino acid sequence variants of the antibodies and/or the binding polypeptides provided herein are contemplated. For example, it may be desirable to improve the binding affinity and/or other biological properties of the antibody and/or binding polypeptide Amino acid sequence variants of an antibody and/or binding polypeptides may be prepared by introducing appropriate modifications into the nucleotide sequence encoding the antibody and/or binding polypeptide, or by peptide synthesis. Such modifications include, for example, deletions from, and/or insertions into and/or substitutions of residues within the amino acid sequences of the antibody and/or binding polypeptide. Any combination of deletion, insertion, and substitution can be made to arrive at the final construct, provided that the final construct possesses the desired characteristics, e.g., target-binding.

In certain embodiments, antibody variants and/or binding polypeptide variants having one or more amino acid substitutions are provided. Sites of interest for substitutional mutagenesis include the HVRs and FRs. Conservative substitutions are shown in Table 1 under the heading of “conservative substitutions.” More substantial changes are provided in Table 1 under the heading of “exemplary substitutions,” and as further described below in reference to amino acid side chain classes Amino acid substitutions may be introduced into an antibody and/or binding polypeptide of interest and the products screened for a desired activity, e.g., retained/improved antigen binding, decreased immunogenicity, or improved ADCC or CDC.

TABLE 1

| | | Preferred |
|---|---|---|
| Original Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al., in METHODS IN MOL. BIOL. 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of the antibody and/or the binding polypeptide that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### G. Antibody and Binding Polypeptide Derivatives

In certain embodiments, an antibody and/or binding polypeptide provided herein may be further modified to contain additional nonproteinaceous moieties that are known in the art and readily available. The moieties suitable for derivatization of the antibody and/or binding polypeptide include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody and/or binding polypeptide may vary, and if more than one polymer is attached, they can be the same or different molecules. In general, the number and/or type of polymers used for derivatization can be determined based on considerations including, but not limited to, the particular properties or functions of the antibody and/or binding polypeptide to be improved, whether the antibody derivative and/or binding polypeptide derivative will be used in a therapy under defined conditions, etc.

In another embodiment, conjugates of an antibody and/or binding polypeptide to nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody and/or binding polypeptide-nonproteinaceous moiety are killed.

### H. Recombinant Methods and Compositions

Antibodies and/or binding polypeptides may be produced using recombinant methods and compositions, e.g., as described in U.S. Pat. No. 4,816,567. In one embodiment, isolated nucleic acid encoding an anti-CDK8 antibody. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid encoding the antibody and/or binding polypeptide are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g., a Chinese Hamster Ovary (CHO) cell or lymphoid cell (e.g., Y0, NS0, Sp20 cell). In one embodiment, a method of making an antibody such as an anti-CDK8 antibody and/or binding polypeptide is provided, wherein the method comprises culturing a host cell comprising a nucleic acid encoding the antibody and/or binding polypeptide, as provided above, under conditions suitable for expression of the antibody and/or binding polypeptide, and optionally recovering the antibody and/or polypeptide from the host cell (or host cell culture medium).

For recombinant production of an antibody such as an anti-CDK8 antibody and/or a binding polypeptide, nucleic acid encoding the antibody and/or the binding polypeptide, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, METHODS IN MOL. BIOL., Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody and/or glycosylated binding polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as YO, NSO and Sp2/0. For a review of certain mammalian host cell lines suitable for antibody production and/or binding polypeptide production, see, e.g., Yazaki and Wu, METHODS IN MOL. BIOL., Vol. 248 (B. K. C. Lo, ed., Humana Press, Totowa, N.J.), pp. 255-268 (2003).

While the description relates primarily to production of antibodies and/or binding polypeptides by culturing cells transformed or transfected with a vector containing antibody- and binding polypeptide-encoding nucleic acid. It is, of course, contemplated that alternative methods, which are well known in the art, may be employed to prepare antibodies and/or binding polypeptides. For instance, the appropriate amino acid sequence, or portions thereof, may be produced by direct peptide synthesis using solid-phase techniques [see, e.g., Stewart et al., Solid-Phase Peptide Synthesis, W.H. Freeman Co., San Francisco, Calif. (1969); Merrifield, J. Am. Chem. Soc., 85:2149-2154 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the antibody and/or binding polypeptide may be chemically synthesized separately and combined using chemical or enzymatic methods to produce the desired antibody and/or binding polypeptide.

### III. Methods of Screening and/or Identifying CDK8 Antagonists with Desired Function

Techniques for generating CDK8 antagonists such as antibodies, binding polypeptides, and/or small molecules have been described above. Additional CDK8 antagonists such as anti-CDK8 antibodies, binding polypeptides, and/or small molecules provided herein may be identified, screened for, or characterized for their physical/chemical properties and/or biological activities by various assays known in the art.

Provided herein are methods of screening for and/or identifying a CDK8 antagonist which promotes cell differentiation said method comprising: contacting a reference cell, wherein the reference cell is a stem cell and/or a cancer stem cell, with a CDK8 candidate antagonist, wherein the CDK8 candidate antagonist binds CDK8, and whereby differentiation of the reference cell into a differentiated cell identifies the CDK8 candidate antagonist as promoting cell differentiation. In some embodiments, the reference cell is a cancer tem cell. In some embodiments, the differentiated cell is a goblet cell and/or enterocyte cell. In some embodiments, the CDK8 candidate antagonist is an antibody, binding polypeptide, small molecule, or polynucleotide. In some embodiments, the CDK8 candidate antagonist induces cancer cell cycle arrest, inhibits cancer cell proliferation, and/or promotes cancer cell death.

Provided herein are methods of screening for and/or identifying a CDK8 antagonist which alters a CDK8 gene signature said method comprising: (a) contacting a reference cell with a CDK8 candidate antagonist, wherein the CDK8 candidate antagonist binds CDK8, (b) determining expression levels of one or more biomarkers of a CDK8 gene signature at one time-point and a second time-point, wherein differential expression levels of one or more biomarkers of a CDK8 gene signature identifies the CDK8 candidate antagonist as a CDK8 antagonist In some embodiments, the CDK8 candidate antagonist is an antibody, binding polypeptide, small molecule, or polynucleotide. In some embodiments, the CDK8 candidate antagonist induces cancer cell cycle arrest, inhibits cancer cell proliferation, and/or promotes cancer cell death.

In some embodiments of any of the articles of manufacture, the one or more biomarkers of the CDK8 gene signature comprises one or more genes listed in Table 2 and/or Table 3. In some embodiments, the one or more genes listed in Table 2 and/or Table 3 comprises one or more ES cell-related genes. In some embodiments, the one or more genes listed in Table 2 and/or Table 3 comprises one or more MYC ES target genes. In some embodiments, the one or more genes listed in Table 2 and/or Table 3 comprises one or more p53 signalling genes, cell cycle genes, Wnt signalling genes, and/or SMAD/BMP signalling genes.

In some embodiments of any of the methods of screening for and/or identifying an CDK8 antagonist, the cancer cell, cancer tissue, or cancer sample is bladder cancer, pancreatic cancer, lung cancer, breast cancer, colon cancer, colorectal cancer, endometrial cancer, head & neck cancer, kidney cancer, ovarian cancer, hypopharyngeal, prostate cancer, esophageal, hepatocellular carcinoma, and/or urinary cancer. In some embodiments of any of the methods of screening for and/or identifying an CDK8 antagonist, the cancer cell, cancer tissue, or cancer sample is from a cancer selected from the group of bladder cancer, pancreatic cancer, lung cancer, breast cancer, colon cancer, colorectal cancer, endometrial cancer, head & neck cancer, kidney cancer, ovarian cancer, and/or urinary cancer. In some embodiments, the cancer cell, cancer tissue, or cancer sample is from a cancer selected from the group of bladder cancer, pancreatic cancer, endometrial cancer, head & neck cancer, kidney cancer, ovarian cancer, and/or urinary cancer.

In some embodiments of any of the methods of screening for and/or identifying an CDK8 antagonist, differential expression levels of one or more biomarkers of a CDK8 gene signature is elevated expression. In some embodiments, elevated expression refers to an overall increase of about any of 10%, 20%, 25%, 30%, 40%, 50%, 60%, 70%, 80%, 85%, 90%, 95%, 96%, 97%, 98%, 99% or greater, in the level of biomarker (e.g., protein or nucleic acid (e.g., gene or mRNA)), detected by standard art known methods such as those described herein, as compared to a reference sample, reference cell, reference tissue, control sample, control cell, or control tissue. In certain embodiments, the elevated expression refers to the increase in expression level/amount of a biomarker in the sample wherein the increase is at least about any of 1.5×, 1.75×, 2×, 3×, 4×, 5×, 6×, 7×, 8×, 9×, 10×, 25×, 50×, 75×, or 100× the expression level/amount of the respective biomarker in a reference sample, reference cell, reference tissue, control sample, control cell, or control tissue. In some embodiments, elevated expression refers to an overall increase of greater than about 1.5 fold, about 1.75 fold, about 2 fold, about 2.25 fold, about 2.5 fold, about 2.75 fold, about 3.0 fold, or about 3.25 fold as compared to a reference sample, reference cell, reference tissue, control sample, control cell, control tissue, or internal control (e.g., housekeeping gene).

In some embodiments of any of the methods of screening for and/or identifying an CDK8 antagonist, differential expression levels of one or more biomarkers of a CDK8 gene signature is reduced expression. In some embodiments, reduced expression refers to an overall reduction of about any of 10%, 20%, 25%, 30%, 40%, 50%, 60%, 70%, 80%, 85%, 90%, 95%, 96%, 97%, 98%, 99% or greater, in the level of biomarker (e.g., protein or nucleic acid (e.g., gene or mRNA)), detected by standard art known methods such as those described herein, as compared to a reference sample, reference cell, reference tissue, control sample, control cell, or control tissue. In certain embodiments, reduced expression refers to the decrease in expression level/amount of a biomarker in the sample wherein the decrease is at least about any of 0.9×, 0.8×, 0.7×, 0.6×, 0.5×, 0.4×, 0.3×, 0.2×, 0.1×, 0.05×, or 0.01× the expression level/amount of the respective biomarker in a reference sample, reference cell, reference tissue, control sample, control cell, or control tissue.

The growth inhibitory effects of a CDK8 antagonist described herein may be assessed by methods known in the art, e.g., using cells which express CDK8 either endogenously or following transfection with the respective gene(s). For example, appropriate tumor cell lines, and CDK8 polypeptide-transfected cells may be treated with a CDK8 antagonist described herein at various concentrations for a few days (e.g., 2-7) days and stained with crystal violet or MTT or analyzed by some other colorimetric assay. Another method of measuring proliferation would be by comparing ³H-thymidine uptake by the cells treated in the presence or absence an antibody, binding polypeptide or small molecule of the invention. After treatment, the cells are harvested and the amount of radioactivity incorporated into the DNA quantitated in a scintillation counter. Appropriate positive controls include treatment of a selected cell line with a growth inhibitory antibody known to inhibit growth of that cell line. Growth inhibition of tumor cells in vivo can be determined in various ways known in the art.

Methods of determining the distribution of cell cycle stage, level of cell proliferation, and/or level of cell death are known in the art and are described in the examples herein. In some embodiments, cancer cell cycle arrest is arrest in G1.

In some embodiments, the CDK8 antagonist will inhibit cancer cell proliferation of the cancer cell, cancer tissue, or cancer sample in vitro or in vivo by about 25-100% compared to the untreated cancer cell, cancer tissue, or cancer sample, more preferably, by about 30-100%, and even more preferably by about 50-100% or about 70-100%. For example, growth inhibition can be measured at a CDK8 antagonist concentration of about 0.5 to about 30 μg/ml or about 0.5 nM to about 200 nM in cell culture, where the growth inhibition is determined 1-10 days after exposure of the tumor cells to the CDK8 candidate antagonist. The CDK8 antagonist is growth inhibitory in vivo if administration of the CDK8 candidate antagonist at about 1 μg/kg to about 100 mg/kg body weight results in reduction in tumor size or reduction of tumor cell proliferation within about 5 days to 3 months from the first administration of the CDK8 candidate antagonist, preferably within about 5 to 30 days.

To select for a CDK8 antagonists which induces cancer cell death, loss of membrane integrity as indicated by, e.g., propidium iodide (PI), trypan blue or 7AAD uptake may be assessed relative to a reference. A PI uptake assay can be performed in the absence of complement and immune effector cells. CDK8-expressing tumor cells are incubated with medium alone or medium containing the appropriate a CDK8 antagonist. The cells are incubated for a 3-day time period. Following each treatment, cells are washed and aliquoted into 35 mm strainer-capped 12×75 tubes (1 ml per tube, 3 tubes per treatment group) for removal of cell clumps. Tubes then receive PI (10 μg/ml). Samples may be analyzed using a FACSCAN® flow cytometer and FACSCONVERT® CellQuest software (Becton Dickinson). Those CDK8 antagonists that induce statistically significant levels of cell death as determined by PI uptake may be selected as cell death-inducing antibodies, binding polypeptides or small molecules.

To screen for CDK8 antagonists which bind to an epitope on or interact with a polypeptide bound by an antibody of interest, a routine cross-blocking assay such as that described in Antibodies, A Laboratory Manual, Cold Spring Harbor Laboratory, Ed Harlow and David Lane (1988), can be performed. This assay can be used to determine if a candidate CDK8 antagonist binds the same site or epitope as a known antibody. Alternatively, or additionally, epitope mapping can be performed by methods known in the art. For example, the antibody and/or binding polypeptide sequence can be mutagenized such as by alanine scanning, to identify contact residues. The mutant antibody is initially tested for binding with polyclonal antibody and/or binding polypeptide to ensure proper folding. In a different method, peptides corresponding to different regions of a polypeptide can be used in competition assays with the candidate antibodies and/or polypeptides or with a candidate antibody and/or binding polypeptide and an antibody with a characterized or known epitope.

In some embodiments of any of the methods of screening and/or identifying, the CDK8 candidate antagonist is an antibody, binding polypeptide, small molecule, or polynucleotide. In some embodiments, the CDK8 candidate antagonist is an antibody. In some embodiments, the CDK8 antagonist is a small molecule.

In one aspect, a CDK8 antagonist is tested for its antigen binding activity, e.g., by known methods such as ELISA, Western blot, etc.

### K. Pharmaceutical Formulations

Pharmaceutical formulations of a CDK8 antagonist as described herein are prepared by mixing such antibody having the desired degree of purity with one or more optional pharmaceutically acceptable carriers (REMINGTON'S PHARMA. SCI. 16th edition, Osol, A. Ed. (1980)), in the form of lyophilized formulations or aqueous solutions. In some embodiments, the CDK8 antagonist is a small molecule, an antibody, binding polypeptide, and/or polynucleotide. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in REMINGTON'S PHARMA. SCI. 16th edition, Osol, A. Ed. (1980).

Sustained-release preparations may be prepared. Suitable examples of sustained-release preparations include semipermeable matrices of solid hydrophobic polymers containing the CDK8 antagonist, which matrices are in the form of shaped articles, e.g., films, or microcapsules.

The formulations to be used for in vivo administration are generally sterile. Sterility may be readily accomplished, e.g., by filtration through sterile filtration membranes.

### L. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a CDK8 antagonist described herein. The label or package insert indicates that the composition is used for treating the condition of choice. Moreover, the article of manufacture may comprise (a) a first container with a composition contained therein, wherein the composition comprises a CDK8 antagonist; and (b) a second container with a composition contained therein, wherein the composition comprises a further cytotoxic or otherwise therapeutic agent.

In some embodiments, the article of manufacture comprises a container, a label on said container, and a composition contained within said container; wherein the composition includes one or more reagents (e.g., primary antibodies that bind to one or more biomarkers or probes and/or primers to one or more of the biomarkers described herein), the label on the container indicating that the composition can be used to evaluate the presence of one or more biomarkers in a sample, and instructions for using the reagents for evaluating the presence of one or more biomarkers in a sample. The article of manufacture can further comprise a set of instructions and materials for preparing the sample and utilizing the reagents. In some embodiments, the article of manufacture may include reagents such as both a primary and secondary antibody, wherein the secondary antibody is conjugated to a label, e.g., an enzymatic label. In some embodiments, the article of manufacture one or more probes and/or primers to one or more of the biomarkers of a CDK8 gene signature described herein.

In some embodiments of any of the articles of manufacture, the one or more biomarkers of the CDK8 gene signature comprises one or more genes listed in Table 2 and/or Table 3. In some embodiments, the one or more genes listed in Table 2 and/or Table 3 comprises one or more ES cell-related genes. In some embodiments, the one or more genes listed in Table 2 and/or Table 3 comprises one or more MYC ES target genes. In some embodiments, the one or more genes listed in Table 2 and/or Table 3 comprises one or more p53 signalling genes, cell cycle genes, Wnt signalling genes, and/or SMAD/BMP signalling genes.

In some embodiments of any of the articles of manufacture, the articles of manufacture comprise primers.

In some embodiments of any of the article of manufacture, the CDK8 antagonist is an antibody, binding polypeptide, small molecule, or polynucleotide. In some embodiments, the CDK8 antagonist is a small molecule. In some embodiments, the small molecule is a small molecule kinase inhibitor. In some embodiments, the small molecule kinase inhibitor is selected from the group consisting of flavopiridol, ABT-869, AST-487, BMS-387032/SNS032, BIRB-796, sorafenib, staurosporine, cortistatin, cortistatin A, and/or a steroidal alkaloid or derivative thereof. In some embodiments, the CDK8 antagonist is an antibody. In some embodiments, the antibody is a monoclonal antibody. In some embodiments, the antibody is a human, humanized, or chimeric antibody. In some embodiments, the antibody is an antibody fragment and the antibody fragment binds CDK8.

The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Other optional components in the article of manufacture include one or more buffers (e.g., block buffer, wash buffer, substrate buffer, etc), other reagents such as substrate (e.g., chromogen) which is chemically altered by an enzymatic label, epitope retrieval solution, control samples (positive and/or negative controls), control slide(s) etc.

It is understood that any of the above articles of manufacture may include an immunoconjugate described herein in place of or in addition to a CDK8 antagonist.

### EXAMPLES

The following are examples of methods and compositions of the invention. It is understood that various other embodiments may be practiced, given the general description provided above.

### Materials and Methods for the Examples

Cell Lines

HT-29 and COLO 205 human colon cancer cells and 293T human embryonic kidney packaging cells were grown in DMEM (high glucose), 10% Fetal Bovine Serum (FBS), and 1% Penicillin-Streptomycin (Invitrogen). R1 mouse embryonic stem (ES) cells (courtesy of Merone Roose-Girma, Genentech), which were derived from a (129×1/SvJ-129S1/SvImJ) F1 mouse embryo (Nagy A. et al., Proc Natl Acad Sci USA 90:8424-8 (1993)) were grown on 0.1% gelatin in the following media: Knockout DMEM (Invitrogen), 15% FBS, 1000 units/ml leukemia inhibitory factor (LIF; Millipore), 5 mM HEPES (MP Biomedicals), 1.4 mM L-Glutamine (MP Biomedicals), 0.05 mM 2-Mercaptoethanol (Sigma), 10 μg/ml Gentamicin (Quality Biological), and 1% Penicillin-Streptomycin (Invitrogen). TC1 ES cells, which were derived from 12956/SvEvTac mice (Deng C. et al., Cell 84:911-21 (1996)), and GSI-1 ES cells, which were derived from 129×1/SvJ mice (Genome Systems), were grown on mitotically inactivated mouse embryonic fibroblast cells (MEFs) in the following media: Knockout DMEM, 15% FBS, 1000 units/ml LIF, 0.1 mM MEM Non-Essential Amino Acids (Gibco), 2 mM L-Glutamine, 0.1 mM 2-Mercaptoethanol, and 1% Penicillin-Streptomycin. To remove MEFs from downstream analyses, TC1 and GSI-1 ES cells were re-plated on 0.1% gelatin prior to analysis. To differentiate the R1 ES cells, LIF was removed from the media and 5 μM retinoic acid (Sigma) was added (Rohwedel, J. et al., Cells Tissues Organs 165, 190-202 (1999)). All cell line stocks are maintained at Genentech and undergo genotyping to verify their identity every six months.

Infection/Transfection Procedures

Short hairpin RNAs (shRNAs) and cDNA expression plasmids were expressed in HT-29, COLO 205, and R1 cells using a lentiviral packaging system. Briefly, 293T cells were transfected with pLKO.1-shRNA vector, pHush-shRNA vector, pHush-cDNA vector, or pLenti6.2-cDNA vector, along with pCMV-VSVG and pCMV-dR8.9 to make replication-incompetent lentiviral particles. Viral particles were added to cells with 5-8 μg/ml polybrene and spin infected at room temperature (1800 rpm, 30-45 minutes). Stable integration of shRNAs was selected with 6-8 μg/ml puromycin (for pLKO.1 R1 knockdown experiments) or with 2 μg/ml puromycin (for pHush xenograft knockdown experiments). Stable integration of cDNAs was selected with 10 μg/ml blasticidin (for pLenti6.2 MYC rescue experiments) or by flow sorting for GFP-positive cells (for pHush CDK8 rescue experiments). HT-29 cells were transiently transfected with siRNAs using Lipofectamine RNAiMAX (Invitrogen).

Xenograft Tumor Models

HT-29 and COLO 205 cells were infected with doxycycline-inducible pHush-shRNAs targeting CDK8 (or NTC control) and selected for stable integration with 2 μg/ml puromycin (Clontech). For each cell line, 5×10⁶ cells were injected subcutaneously into the backs of 26 female NCr nude mice (Taconic) to initiate tumor growth. The size of each tumor was measured using a calliper. Once tumors reached 225 mm³, the animals from each cell line were split into two groups. For each cell line, the first group of 13 mice was fed 5% sucrose in their water (control group) while the second group of 13 mice was fed 5% sucrose+1 mg/ml doxycycline (Clontech) to induced hairpin expression. After 8 days (HT-29) or 12 days (COLO 205), three of the mice from each group were euthanized and the tumors were harvested for Western blot analysis. The remaining 10 mice per group were monitored until Day 16, and the tumor volume was measured every 3-4 days. In parallel, the weight of the mice was also measured and recorded. Tumor growth inhibition values were determined by an area under the curve calculation.

Human Colon Tissue Samples

Frozen normal human colon, colon tumors, and metastatic colon tumors were obtained from Asterand, Integrated Laboratory Services, Cooperative Human Tissue Network, or ProteoGenex. Prior to Western blot analysis, each tumor was verified by a board certified pathologist (R.F.) to contain a high percentage of tumor cells.

Plasmids and RNAi Constructs

Human CDK8 cDNA (Origene) was cloned into pAcGP67 vector (BD Biosciences) that contained an N-terminal FLAG tag. FLAG-tagged CDK8 was PCR amplified and cloned into pSHUTTLE-CMV-TO and then Gateway recombined (Invitrogen) into pHush-GFP expression vector (Gray, D. C. et al., BMC Biotechnol 7, 61 (2007)). Human MYC cDNA (Invitrogen) was cloned into pLenti6.2 vector by Gateway recombination (Invitrogen). The T58A and S62A mutations were introduced by QuikChange site directed mutagenesis kit (Agilent/Stratagene) and verified by sequencing.

For the xenograft studies, a doxycycline-inducible pHush-shRNA system was utilized as described in Gray et al., BMC Biotechnol. 7, 61 (2007). The pHush-shNTC control was obtained from David Davis (Genentech). The shCDK8 targeting sequence (GAATGGTGAAGTCACTATTAT (SEQ ID NO:5)) was first cloned into the pSHUTTLE-H1 vector. Then the pSHUTTLE-H1-shRNA was Gateway (Invitrogen) recombined into a puromycin-selectable pHush vector (Gray, D. C. et al., BMC Biotechnol 7, 61 (2007)). For the R1 ES cell experiments, the following shRNA target sequences in pLKO.1 vector were utilized (from Open Biosystems unless otherwise stated): shNTC, CAACAAGATGAAGAGCACCAA (Sigma (SEQ ID NO:6); shCdk8-1, CCCGATTATTTAATTCACCTT (SEQ ID NO:7); shCdk8-2, CAGGGATTTGAAACCTGCTAA [mouse-specific] (SEQ ID NO:8); shNanog, GCCAGTGATTTGGAGGTGAAT (SEQ ID NO:9); shMed12-1, CCTCTCCCTTTGATGATCCTA (SEQ ID NO:10); shMed12-2, CCGTGCGATTACCAATGCAAA (SEQ ID NO:11). For the HT-29 colon cancer experiments, the following siRNA target sequences were utilized (from Ambion): siNTC (Negative Control #1); siCDK8-1, CAAAACTAGTAATCCTTATTT (SEQ ID NO:12); siCDK8-2, CCCTTACCCAAAACGAGAATT (SEQ ID NO:13).

Antibodies

The following antibodies were utilized: ACTIN (clone C4; MP Biomedicals), CDK8 (clone C-19; Santa Cruz Biotechnology), NANOG (Millipore), OCT4 (Abcam), c-MYC (clone D84C12; Cell Signaling Technology), c-MYC-pT58 (Sigma), c-MYC-pS62 (Abcam), c-MYC-pT58/S62 (Abcam), Alexa Fluor 488 donkey anti-rabbit IgG (Invitrogen), Alexa Fluor 568 donkey anti-goat IgG (Invitrogen), CD44-PE/Cy5 (Biolegend), and CD133-PE (Miltenyi Biotec).

Histological, Immunohistochemical, and FACS Analyses

HT-29 xenograft tumors were stained for alcian blue as described in Sheehan, Dezna C. and Hrapchak, Barbara B., THEORY AND PRACTICE OF HISTOTECHNOLOGY, 2d ed. (Mosby, St. Louis, 1980). Hematoxylin and eosin stained xenograft tumors analysis were performed by a clinical pathologist (R.F.) to determine the differentiation status Immunohistochemistry of CDK8 was performed as previously described in Firestein, R. et al., Nature 455, 547-551 (2008). R1 ES cells were stained for alkaline phosphatase activity using an alkaline phosphatase detection kit (Millipore). To quantify the ES cell colonies, alkaline phosphatase positively stained colonies were manually counted under a low magnification microscope (each field was 24 mm²). A minimum of four different fields were counted and then averaged. For immunofluorescence, cells were grown in 96-well, black-walled plates. Cells were fixed with 4% paraformaldehyde for 5 min. and permeabilized/blocked with PBS containing 10% normal horse serum, 0.1% Triton X-100. Primary antibody was added for 1 hour followed by secondary antibody for 30 minutes. Hoechst 33342 (Invitrogen) was added for 5 min. to stain nuclei. For FACS analysis, xenograft tumor cells were dissociated with collagenase for 30 minutes, washed in PBS+2% FBS, stained 10 minutes for CD133, CD44, and a mouse lineage antibody panel (BD Biosciences) to exclude mouse cells, and analyzed on a FACSCalibur flow cytometer (BD Biosciences).

Gene Expression Analysis

For quantitative RT-PCR, total RNA was isolated with the RNeasy mini kit (Qiagen). Reverse transcription followed by quantitative PCR was performed with the TaqMan one-step RT-PCR master mix using Taqman gene-specific probes (Applied Biosystems).

Microarray Hybridization

For microarray studies, total RNA was harvested from cells in triplicate using RNeasy mini kit with on-column DNase digestion (Qiagen). For HT-29 cells, RNA was harvested three days after siRNA transfection. For R1 cells, RNA was harvested at Day 8 or Day 13 after shRNA infection. RNA was quantified using UV-spec Nanodrop (Thermo Scientific) and then profiled on Agilent Bioanalyzer. RNA was amplified and hybridized to whole human or mouse genome 4×44K gene expression arrays according to manufacturer protocol (Agilent). Universal human or mouse reference RNA (Agilent/Stratagene) was used as reference control.

Microarray Data Analysis

CDK8-Regulated Genes in HT-29 Cells

The microarray data output was a ratio of the sample RNA to the reference control RNA. Only genes with data in at least 70% of experiments were analyzed. Microarray data was log₂-transformed, mean centered, then zero-transformed on the siNTC samples. Genes that significantly changed upon siCDK8 in HT-29 cells were identified by carrying out a Student's t-test between all siNTC controls and all replicates of siCDK8-1/-2. The top 1500 induced or repressed genes/probes were selected (P=0.001, Student's t-test between siNTC and two independent CDK8 siRNAs).

CDK8-Regulated Genes in R1 ES Cells

The microarray data was processed in the same way as the HT-29 cells described above. The top 1500 genes/probes significantly changing upon shCdk8 at Day 8 in R1 cells were identified as described above for HT-29 cells (P=0.003, Student's t-test between shNTC and two independent CDK8 shRNAs).

Gene Set Enrichment/Pathway Analysis

Gene set enrichment analysis was carried out using Genomica (http://genomica.weizmann.ac.il/) as described in Segal, E. et al., Nat Genet. 36, 1090-1098 (2004). P-values were determined by the hypergeometric distribution. The following gene sets were used: gene ontology (Ashburner, M. et al., Nat Genet. 25, 25-29 (2000)), chromatin immunoprecipitation-microarray target gene sets for mouse ES cell transcription factors (Kim, J et al., Cell 132, 1049-1061 (2008)); and mouse ES cell-related and adult stem cell-related gene signatures (Wong, D. J. et al., Cell Stem Cell 2, 333-344 (2008)). Enrichment analysis for signalling pathways was carried out using Ingenuity Pathway Analysis (Ingenuity Systems, www.ingenuity.com). P-values were determined using a fisher exact test.

Comparison to MED12 Knockdown Data

Focusing on the mediator component MED12-regulated gene as defined by Kagey et al., Nature 467, 430-435 (2010), the gene expression pattern was compared to data following CDK8 or MED12 knockdown in ES cells, as well as to data following forced differentiation of ES cells and NANOG or OCT4 knockdown in ES cells from Gene Expression Omnibus accession GSE4189; Loh, Y. H. et al., Nat Genet. 38, 431-440 (2006)). To determine the similarity of the expression patterns, a Pearson correlation was calculated between the shMed12 expression pattern from Kagey, M. H. et al., Nature 467, 430-435 (2010) and the expression pattern from all other data sets.

Correlation of HT-29 CDK8-Regulated Signature to Individual Genes in Colon Cancer

The expression pattern of the top 1500 genes that change upon siCDK8 in HT-29 human colon cancer cells (the HT-29 CDK8-regulated signature) was collapsed into a single expression value for each gene by subtracting the average log₂ expression value of each gene in siCDK8 samples from the average log₂ expression value in siNTC samples. CDK8-induced genes were positive values and CDK8-repressed genes were negative values. Expression of this signature was then correlated to the expression of individual genes in two primary human colon cancer expression data sets: 100 tumors from Gene Expression Omnibus accession GSE5206 and 130 tumors from Gene Logic. First, a Pearson correlation was calculated between the collapsed HT-29 CDK8-regulated signature and the expression values for these genes in each tumor. This was essentially a score for the level of expression of the CDK8-regulated signature in each tumor, where a high correlation value indicates high expression of the CDK8-induced/repressed expression signature. Then a second Pearson correlation was calculated for each tumor between the Pearson value obtained in the first step and the expression of individual genes. High correlation values indicated a concordance between expression of the gene with expression of the CDK8-regulated signature.

CDK8-Induced MYC ES Cell Target Gene Expression in Colon Tumors

For comparison to CDK8 expression, CDK8-induced MYC ES cell target genes from HT-29 cells were selected out of microarray data from 227 primary human colon cancer tumors (Gene Logic). The average log₂ expression of the CDK8-induced MYC ES targets was calculated for each tumor, and the tumors were sorted from high to low average target gene expression (for comparison, the average expression of all MYC ES cell targets (Kim, J. et al., Cell 132, 1049-1061 (2008)) in each tumor was determined). The tumors were split into two groups by high versus low target gene expression, and CDK8 expression levels in each group were averaged. For correlation to differentiation status, CDK8-induced MYC ES cell target genes were selected out of microarray data from 213 primary human colon tumors that had known differentiation status (Gene Expression Omnibus accession GSE17538; (Smith J J. et al., Gastroenterology 138:958-68 (2010)). The average expression of the targets was calculated for each tumor, and the tumors were sorted from high to low expression (the same procedure was also carried out for all MYC ES targets). The tumors were split into two groups by high versus low target gene expression, and the number of poorly differentiated tumors in each group was counted. The enrichment of poorly differentiated tumors in one group over the other was calculated with a fisher exact test using a 2×2 contingency table. For correlation to clinical outcome, the same procedure described above was carried out on 50 tumors that had undergone recurrence (Gene Expression Omnibus accession GSE14333; Jorissen R. N. et al., Colorectal Cancer. Clin Cancer Res 15:7642-51 (2009)). After splitting the tumors into high versus low expression of CDK8-induced MYC ES targets, the average time to recurrence was calculated for each group. For comparison, the same process was carried out for all MYC ES cell targets.

### Example 1

### Characterization of CDK8 Loss on Tumor Growth and Gene Expression

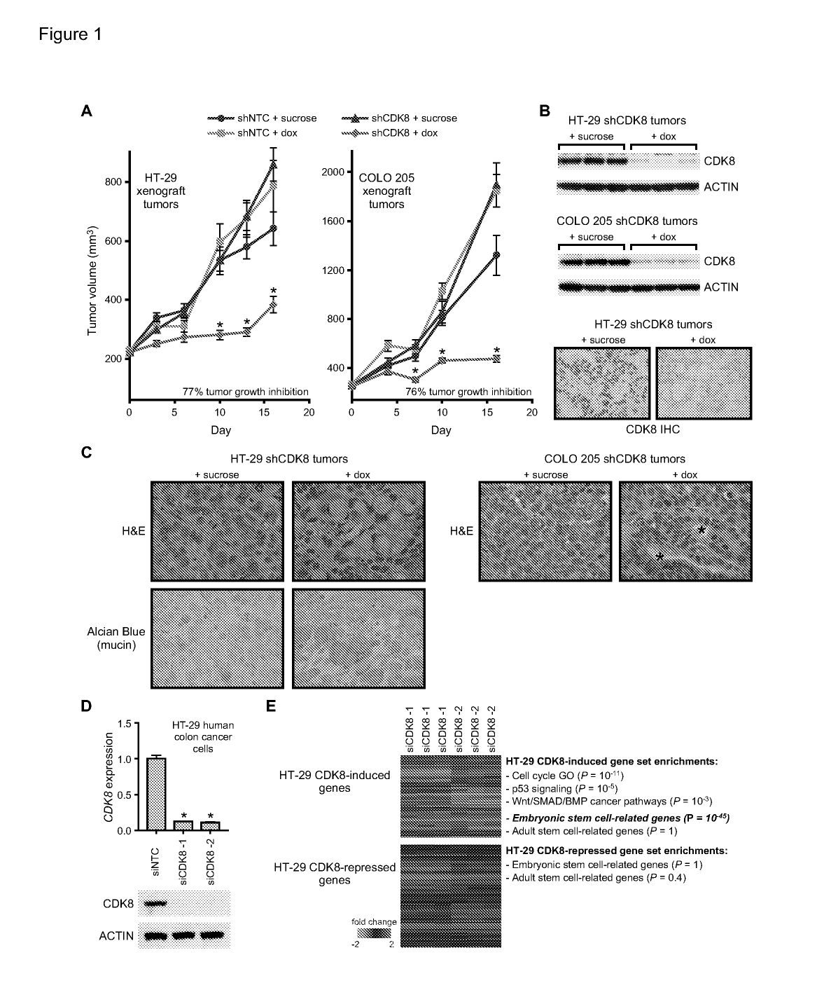

To characterize the effect of acute loss of CDK8 on tumor growth in vivo, an inducible short hairpin RNA (shRNA) system (Hoeflich, K. P. et al., Cancer Res 66, 999-1006 (2006)) was used to deplete endogenous CDK8 in fully formed tumors. shRNAs to CDK8 and a non-targeting control (shNTC) were introduced into two human colon cancer cell lines (HT-29 and COLO 205) and grown as xenograft tumors. These cell lines harbour genomic copy number gain and overexpression of CDK8 and were sensitive to CDK8 loss in vitro. Firestein, R. et al., Nature 455, 547-551 (2008). Xenograft tumor volume was measured over time (n=10 mice per group). The tumor growth inhibition values were determined by an area under the curve calculation. As shown in FIG. 1A, doxycycline-induced acute knockdown of CDK8 protein in fully formed tumors led to profound growth inhibition in both HT-29 and COLO 205 xenograft tumors when compared to either the shNTC controls and the non-doxycycline induced shCDK8 tumors. No significant weight changes were observed throughout the duration of the study for any of the treatment groups, consistent with the notion that loss of CDK8 in the tumor itself was causing growth inhibition (data not shown). Knockdown of CDK8 in the tumors after doxycycline treatment was confirmed by both Western blot and immunohistochemistry (FIG. 1B).

Initial immunohistochemical analyses revealed that HT-29 tumor cells with depleted CDK8 showed histological changes characterized by the formation of large cytoplasmic inclusions. Further morphological examination of these tumors showed that while the HT-29 and COLO 205 models normally grow as sheets of cells characteristic of poorly differentiated tumors, loss of CDK8 led to a well-differentiated tumor state in both tumor models (FIG. 1C). CDK8 depletion led to accumulation of mucin rich deposits in HT-29 xenografts, consistent with goblet cell differentiation, and led to well-formed glands with evidence of polarization in COLO 205 xenografts, consistent with enterocyte differentiation. In contrast, when CDK8 loss was induced in SW837 tumors, a colon cancer xenograft characterized by lack of CDK8 amplification and lower protein expression (Firestein R. et al., Nature 455:547-51 (2008)), little effect on tumor growth and differentiation was seen (data not shown).

It has been proposed that colon tumor growth may be maintained by a small population of “cancer stem cells” (Clarke M. F. et al., Cell 124:1111-5 (2006)). However, as shown herein, CDK8 was widely expressed in all xenograft tumor cells (FIG. 1B), mimicking the broad expression pattern of CDK8 in primary colon tumors (Firestein R. et al., Int J Cancer 126:2863-73 (2010)). Further, CDK8 inhibition in xenograft tumors and in culture had little effect on the levels of the proposed colon cancer stem cell makers CD133 and CD44 (O'Brien C. A. et al., Nature 445:106-10 (2007); Ricci-Vitiani L. et al., Nature 445:111-5 (2007); Dalerba P. et al., Proc Natl Acad Sci USA 104:10158-63 (2007)) (data not shown). Together these observations demonstrate that CDK8 was required for tumor growth and maintenance of a de-differentiated state in vivo.

To gain insight into potential mechanisms for CDK8-mediated regulation of tumor growth and differentiation, the primary gene expression changes that occur after CDK8 knockdown in HT-29 cells was accessed using two independent small interfering RNAs (siRNAs) (FIG. 1D). The expression of 1500 genes were changed in CDK8 depleted cells compared to the siNTC control, which included genes that were enriched in pathways implicated in CDK8 biology (p53 signalling (Donner, A. J. et al., Mol Cell 27, 121-133 (2007)), cell cycle, Wnt signalling (Firestein, R. et al., Nature 455, 547-551 (2008); Morris, E. J. et al., Nature 455, 552-556 (2008) and SMAD/BMP signalling (Alarcon, C. et al., Cell 139, 757-769 (2009)); FIG. 1E and Table 2).

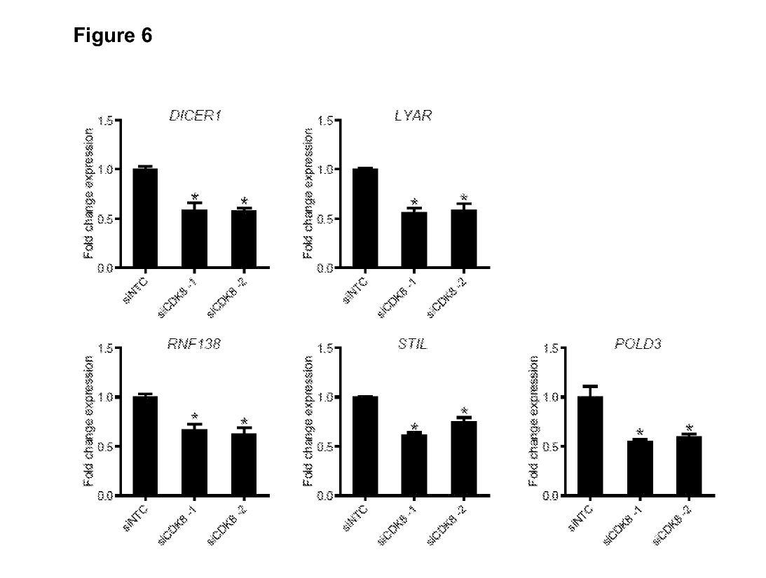

Given the effect of CDK8 loss on tumor differentiation (FIG. 1C), enrichment of defined embryonic stem cell-related and adult stem cell-related gene sets derived by integrating over 100 different expression profiles of a wide array of stem cells (Wong, D. J. et al., Cell Stem Cell 2, 333-344 (2008)) was evaluated. CDK8-induced genes were specifically enriched for ES cell-related genes, but not for adult stem cell-related genes (FIG. 1E). This was unique to CDK8-induced genes, as CDK8-repressed genes showed no enrichment for ES or adult stem cell-related genes. Quantitative RT-PCR confirmed the reduced expression of multiple ES cell-related genes after CDK8 knockdown (FIG. 6). These observations indicated that CDK8 positively regulates an ES cell gene expression program in colon cancer cells and suggested a common role for CDK8 function in ES and cancer cells.

### Example 2

### Characterization of CDK8 in Embryonic Stem (ES) Cells

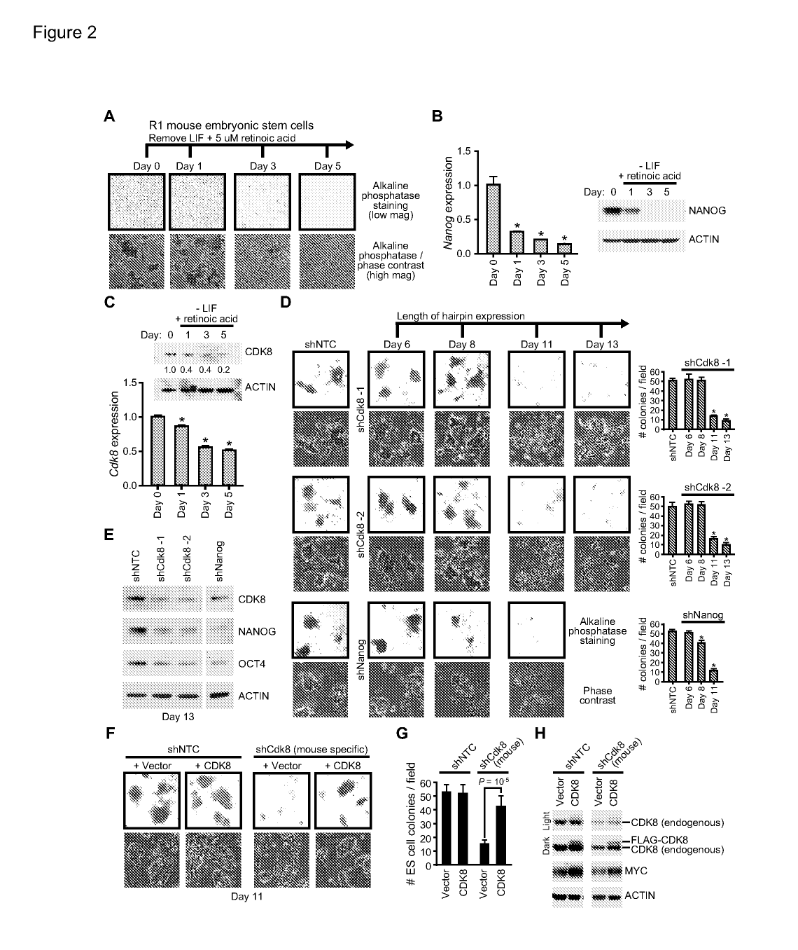

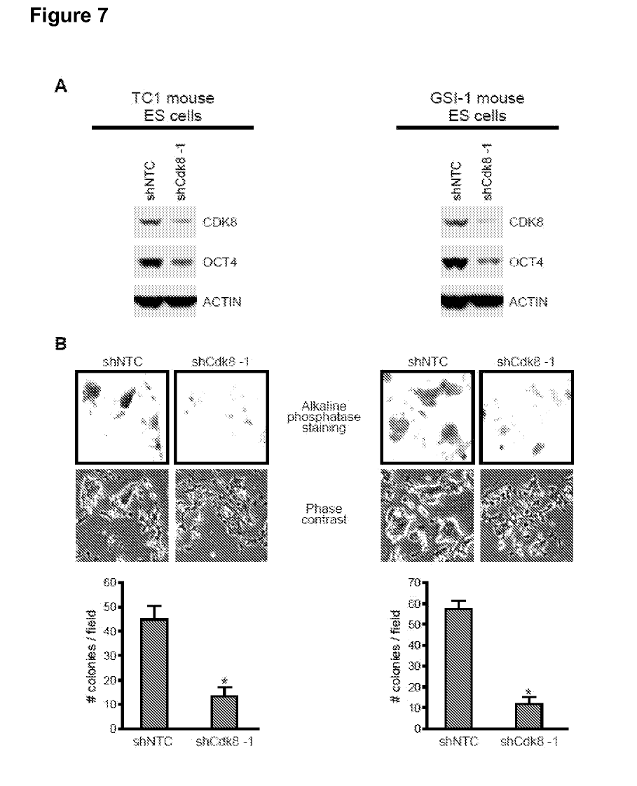

To directly test this hypothesis, CDK8 expression was characterized in murine ES cells subjected to forced differentiation by removal of leukaemia inhibitory factor (LIF) and addition of retinoic acid (Rohwedel, J. et al., Cells Tissues Organs 165, 190-202 (1999)). Loss of ES pluripotency was marked by reduced alkaline phosphatase staining (Pease, S. et al., Dev Biol 141, 344-352 (1990)) and loss of expression of the ES cell core regulator NANOG (FIG. 2A, B). Concomitant with ES cell differentiation, that CDK8 levels were reduced at both the mRNA and protein level (FIG. 2C). To determine whether CDK8 was directly required to maintain ES cells in an undifferentiated state, murine ES cells were treated with shCdk8 or positive (shNanog) and negative (shNTC) controls. Loss of CDK8 in ES cells led to a significant reduction in ES cell pluripotency as evidenced by reduced alkaline phosphatase staining, reduced ES cell colony formation, and reduced NANOG and OCT4 protein levels 11 days after shRNA treatment (FIG. 2D, E, and data not shown). CDK8 inhibition in two additional murine ES cell lines, TC1 and GSI-1, also significantly reduced ES cell pluripotency (FIG. 7). All three ES cell lines analyzed had a normal karyotype (data not shown) and were disomic for Cdk8 copy number (data not shown). To determine whether the observed ES cell differentiation was mediated directly by CDK8 and not an off-target effect, we rescued the RNAi phenotype by simultaneously expressing human CDK8 in ES cells treated with mouse-specific shCdk8. Expression of CDK8 was sufficient to prevent the cells from undergoing shCdk8-induced differentiation (FIG. 2F-H). These data indicate that CDK8 is required to maintain ES cells in an undifferentiated state, and similar to the observation in the tumor models, reduced CDK8 expression promotes differentiation.

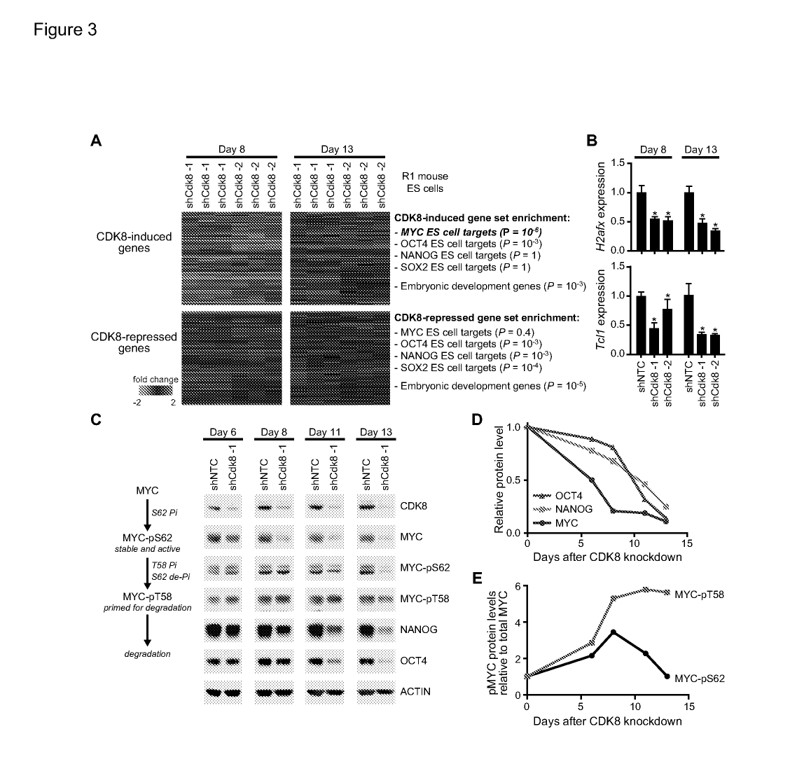

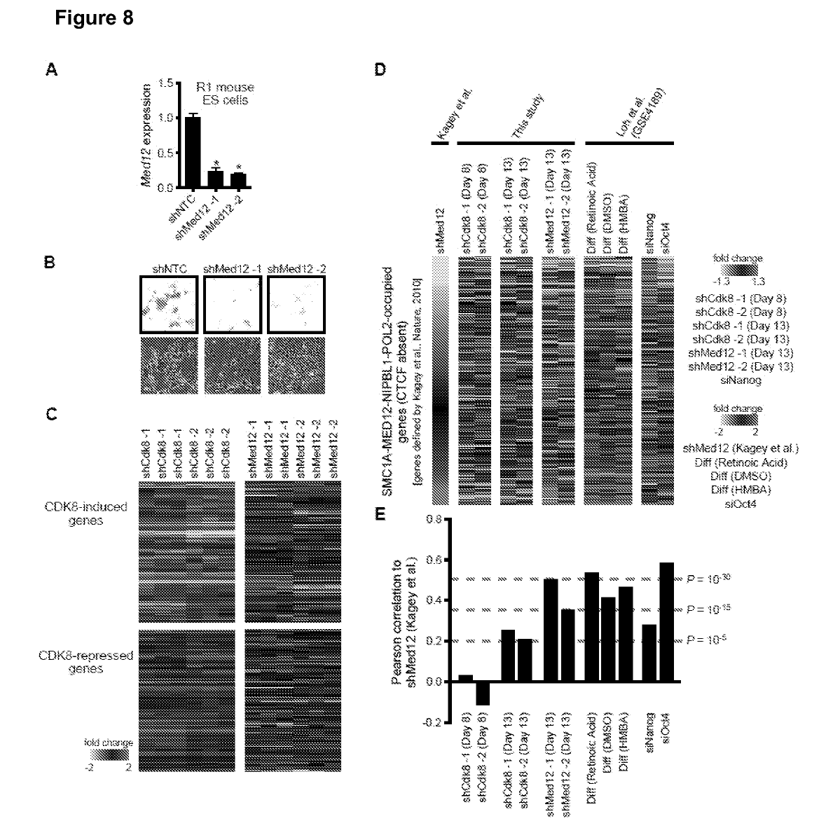

To determine which transcriptional pathways CDK8 regulates in ES cells, gene expression analysis was conducted following CDK8 loss in R1 mouse ES cells both prior to the onset of differentiation (Day 8) and after differentiation (Day 13). The top 1500 genes that significantly changed upon CDK8 loss prior to differentiation at Day 8 were identified (FIG. 3A and Table 3). Consistent with its observed effects on ES cell pluripotency, both CDK8-induced and CDK8-repressed gene signatures identified at the onset of differentiation (Day 8) were enriched for genes involved in ES cell function (FIG. 3A). Reduced expression of a subset of these ES cell-related genes (Andang, M. et al., Nature 451, 460-464 (2008); Glover, C. H. et al., PLoS Comput Biol 2, e158 (2006)) after CDK8 knockdown was confirmed by quantitative RT-PCR (FIG. 3B). CDK8-regulated genes maintained a very similar expression pattern post differentiation at Day 13 (FIG. 3A), suggesting that the gene expression program introduced prior to differentiation remained present after differentiation occurred. In contrast, the expression pattern of ES cells depleted of the Mediator component MED12 was distinct from CDK8 knockdown cells (FIG. 8), suggesting that CDK8 and MED12 regulate ES cell pluripotency via distinct mechanisms.

In ES cells, a small number of core transcription factors (NANOG, OCT4, SOX2, and c-MYC) and their downstream target genes were essential for maintaining the proliferative capacity and pluripotent state of ES cells. Young R A. Cell 144:940-54 (2011); Cartwright, P. et al., Development 132, 885-896 (2005); Chambers, I. & Smith, A., Oncogene 23, 7150-7160 (2004). Target genes for NANOG, OCT4, and SOX2, identified through genome-wide chromatin immunoprecipitation experiments in mouse ES cells (Kim, J et al., Cell 132, 1049-1061 (2008)), showed weak enrichment for CDK8-regulated genes in ES cells, while target genes for c-MYC (referred to as MYC from here on) were more strongly enriched (FIG. 3A). Specifically, MYC ES cell targets were strongly enriched in CDK8-induced genes but not in CDK8-repressed genes. This suggested that CDK8 may regulate target gene expression of core transcription factors in ES cells by promoting MYC target gene expression.

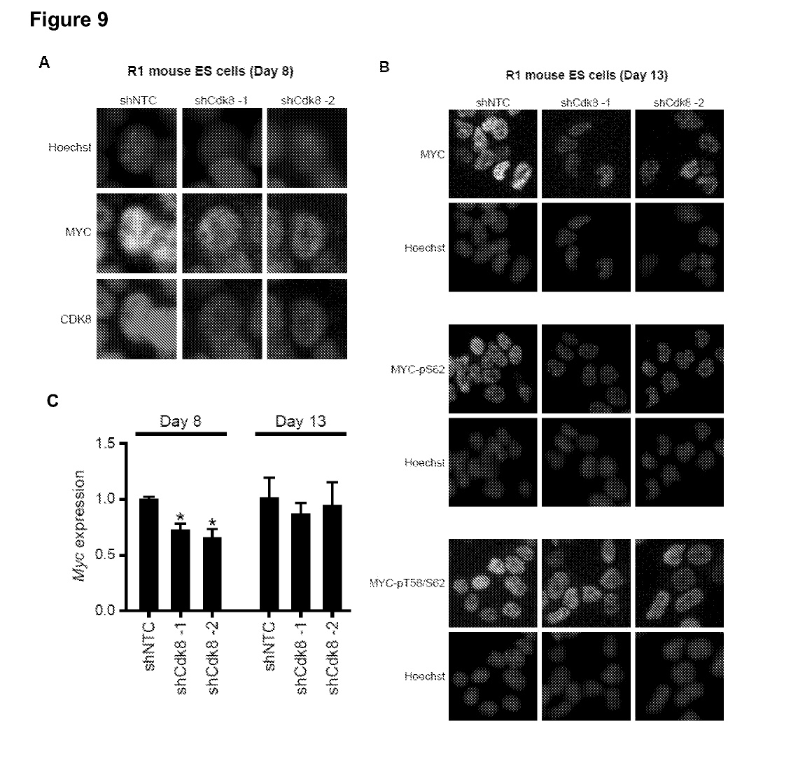

To dissect the temporal relationship between CDK8 loss and the transcriptional output from MYC, OCT4 and NANOG, the expression of these essential transcriptional factors was examined at multiple time points before, during, and after the ES cells underwent CDK8-loss induced differentiation. MYC levels were specifically reduced (Days 6, 8) well before either phenotypic changes of differentiation or changes in NANOG and OCT4 levels were observed (FIG. 3C, D). Myc mRNA levels were either weakly reduced (Day 8) or unchanged (Day 13) upon CDK8 loss (FIG. 9C), suggesting that MYC was regulated by post-transcriptional mechanisms. A critical step in regulating MYC activity involves priming the protein for degradation or transcriptional activation by phosphorylation on threonine 58 (T58) and serine 62 (S62), respectively (Sears, R. et al., Genes Dev 14, 2501-2514 (2000); Sears, R. C., Cell Cycle 3, 1133-1137 (2004)). Using phospho-specific antibodies to both T58-MYC and S62-MYC, a relative increase in the proportion of the unstable T58-phospho-specific MYC was found and a decrease in the active S62-phospho-specific MYC after CDK8 depletion (FIG. 3C, E and FIG. 9A, B). Conversely, overexpression of CDK8 in either shNTC or shCdk8 treated ES cells increased MYC protein levels (FIG. 2H). These data suggest that CDK8 regulates ES pluripotency by maintaining sufficient levels of active MYC protein, which in turn can alter the expression levels of specific MYC target genes.

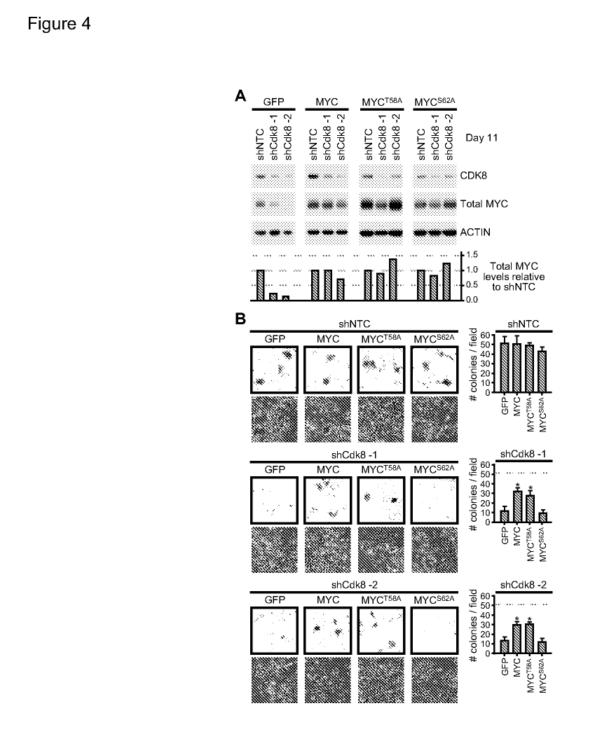

Next the sufficiency of MYC for CDK8-mediated ES cell pluripotency was examined Wildtype MYC, degradation resistant MYC^{T58A}, or inactive MYC^{S62A} was expressed in conjunction with CDK8 knockdown, and the ability of MYC protein levels was able to restore and rescue the differentiation phenotype caused by loss of CDK8. Exogenous expression of either wildtype MYC or MYC^{T58A} in ES cells, which increased MYC levels to that seen in control shNTC cells, partially rescued the loss of ES cell pluripotency imparted by CDK8 depletion (FIG. 4A, B). In contrast, expression of MYC^{S62A}, which disrupts the active phosphorylation site, increased total MYC levels but was unable to rescue the defect in pluripotency. These data reveal that CDK8 regulation of ES cell pluripotency was mediated through MYC.

### Example 3

### Further Characterization of CDK8 Loss in Tumor Cells

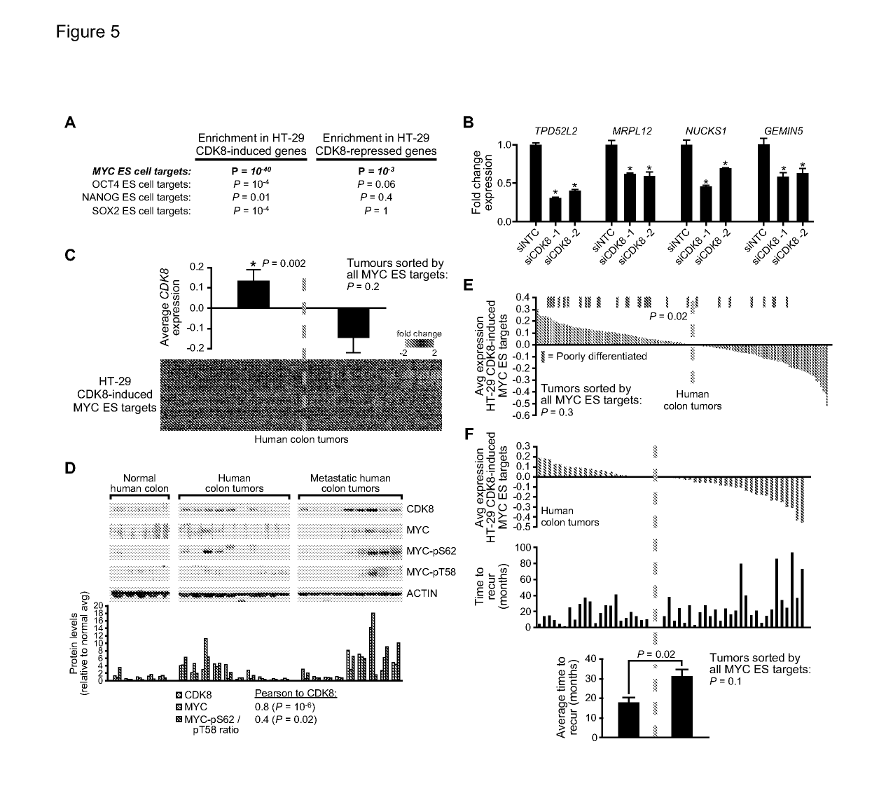

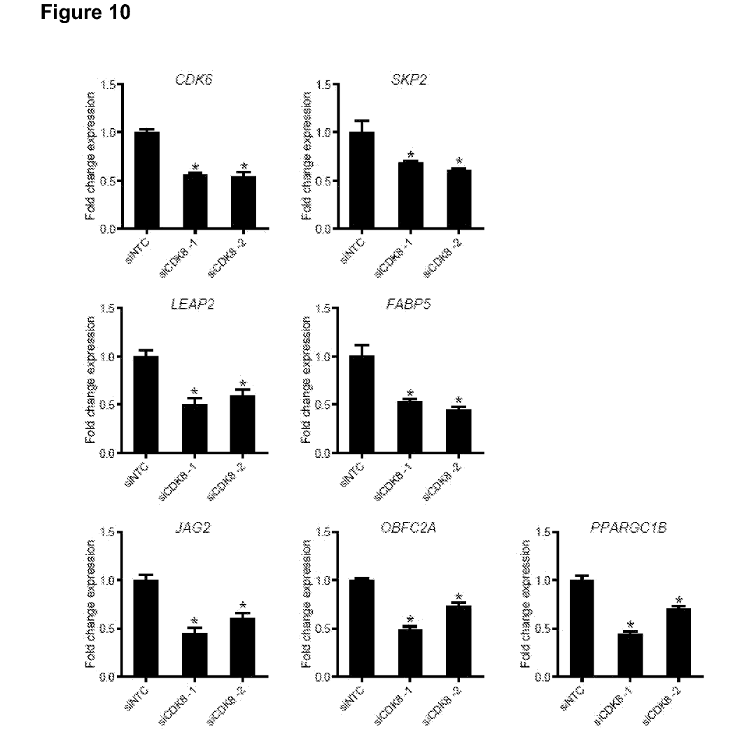

To determine whether a common genetic circuitry underlies the ability of CDK8 to regulate both ES cell pluripotency and cancer, the effect of loss of CDK8 in human colon cancer cells on ES cell transcription factor related gene expression was evaluated. Similar to our findings in ES cells, CDK8-induced genes in colon cancer cells were more strongly enriched for MYC-mediated ES cell target genes than for OCT4, NANOG, and SOX2 ES cell targets (FIG. 5A). Quantitative RT-PCR confirmed the reduced expression of multiple MYC ES cell target genes after CDK8 knockdown in these colon cancer cells (FIG. 5B and FIG. 10). The data implied that CDK8 regulates a specific set of ES cell-related MYC target genes in both colon cancer and embryonic stem cells.

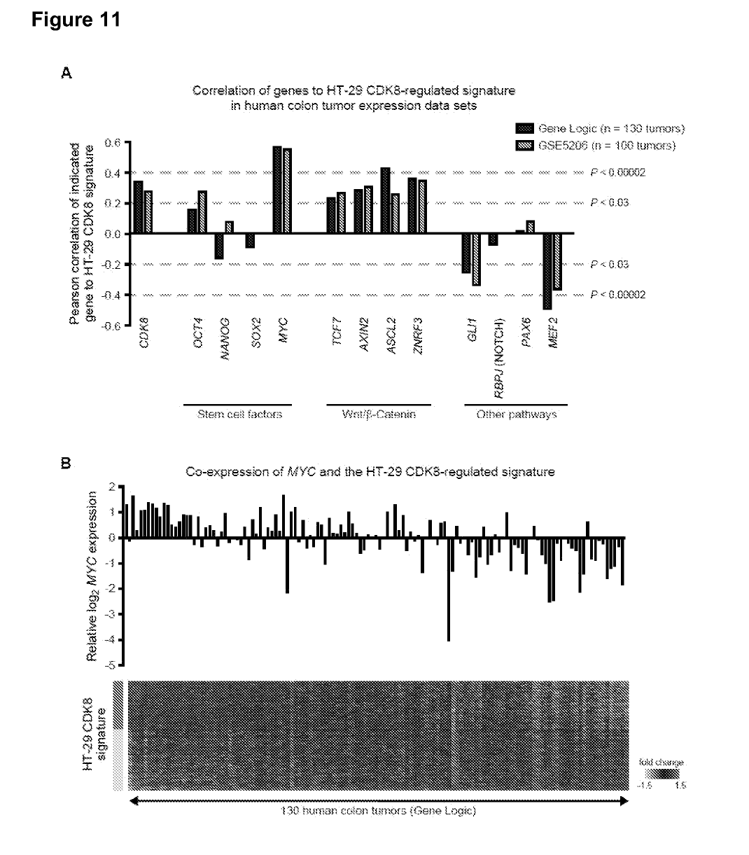

To characterize the interplay between CDK8 and MYC in human colon tumors, two independent cohorts of 100 and 130 human colon cancers were analyzed. Consistent with the observation that MYC targets were regulated by CDK8 in HT-29 colon cancer cells (FIG. 5A), increased MYC expression in both cohorts of human tumors was strongly associated with the presence of the HT-29 CDK8-regulated gene signature (FIG. 11). MYC overexpression can confer stem cell-like properties to epithelial cancer cells (Wong, D. J. et al., Cell Stem Cell 2, 333-344 (2008)), and a MYC-centric gene expression program was found to be similarly expressed in both ES cells and multiple tumor types. Kim, J. et al., Cell 143, 313-324 (2010). To determine whether CDK8 specifically regulates the subset of MYC target genes important for ES cell pluripotency in human tumors, the expression of the CDK8-induced MYC ES cell target genes was evaluated (identified in FIG. 5A and listed in Table 2). High CDK8 levels correlated with increased expression of the CDK8-induced MYC ES cell targets in colon tumors; in contrast, expression of the whole set of MYC ES target genes (Kim, J. et al., Cell 132, 1049-1061 (2008)) was not associated with high CDK8 levels (FIG. 5C). Consistent with this, high CDK8 protein expression in primary and metastatic colon tumors was characterized by increased total and active S62-phosphorylated MYC when compared to the unstable T58-phosphorylated MYC (FIG. 5D). These data implied that the ability of CDK8 to regulate MYC in ES cells extends to human tumors as well.

Genetic signatures related to ES cell pluripotency have been found to predict high tumor grade and poor clinical outcome in several cancer types. Ben-Porath, I. et al., Nat Genet. 40, 499-507 (2008); Wong, D. J. et al., Cell Stem Cell 2, 333-344 (2008); Kim, J et al., Cell 143, 313-324 (2010). Consistent with these observations that CDK8 expression was important for maintaining tumors in a poorly differentiated state in vivo (FIG. 1C) and ES cells in an undifferentiated state (FIG. 2D), the CDK8-induced MYC ES cell signature was enriched in colon tumors characterized by both poor differentiation and poor patient outcome (FIG. 5E, F). Notably, this effect was CDK8 specific as signatures that include all MYC ES cell target genes were not found to be strongly associated with either tumor grade or patient survival (FIG. 5E, F). These data showed that CDK8 regulation of a MYC-centric ES cell signature was active and clinically defines a subset of colon cancers with poor differentiation and poor prognosis.

Here a novel role for the CDK8 oncogene in regulating tumor differentiation and stem cell Pluripotency has been found. Specifically, in xenograft tumor models CDK8 was required to promote rapid tumor growth as well as maintain the tumors in an undifferentiated state. Similarly, CDK8 was highly expressed in ES cells and was required to maintain ES cells in an undifferentiated, pluripotent state. CDK8 regulates MYC protein levels and MYC target gene expression to promote ES cell pluripotency, and expression of CDK8-regulated MYC target genes was predictive of tumor differentiation and clinical outcome of primary human colon tumors.

Recent studies have identified a role for Mediator components in regulating ES cell pluripotency. In ES cells, the Mediator component MED12 binds to the master ES cell regulator NANOG, and MED12 and NANOG were found to co-occupy and regulate the expression of specific NANOG target genes. Tutter, A. V. et al., J Biol Chem 284, 3709-3718 (2009). And recently, multiple Mediator components, including MED12, were found to interact with cohesin at many target genes in ES cells to regulate their expression and modulate ES cell Pluripotency. Kagey, M. H. et al., Nature 467, 430-435 (2010). The expression pattern of ES cells depleted of the Mediator component MED12 was found distinct from CDK8 knockdown cells. This implies that in ES cells, CDK8 and MED12 act divergently to regulate ES cell pluripotency through different mechanisms and the unique Mediator-independent functions of CDK8.

The finding that CDK8 regulates MYC at the protein levels raises an important distinction however been cancer and ES cell biology. Previous work in colon cancer cells revealed that CDK8 inhibition reduced both MYC mRNA and protein levels, suggesting that CDK8 regulates MYC on a transcriptional level. Firestein, R. et al., Nature 455, 547-551 (2008). In stem cells, however, CDK8 inhibition had little effect on MYC transcript levels but strongly reduced MYC protein levels and altered the MYC post-translational modification landscape. Thus in cancer cells and in stem cells, CDK8 may regulate MYC through distinct mechanisms. MYC was known to undergo extensive post-translational modifications from a multitude of inputs, including other CDK proteins (Vervoorts J. et al., J Biol Chem 281:34725-9 (2006); Hann S R. Semin Cancer Biol 16:288-302 (2006)). While the CDK8-MYC connection in stem cells was important to maintain pluripotency, it was unknown whether CDK8 was directly acting on MYC (such as through phosphorylation of S62 or other residues) or though indirect mechanisms on MYC or MYC target genes.

While the data imply that CDK8 regulates MYC activity, alternatively it is plausible that CDK8 and MYC may function convergently yet independently to regulate ES cell gene expression. For example, MYC regulation of RNA polymerase II pause release at ES cell target genes (Rahl P. B. et al., Cell 141:432-45 (2010)) could act in tandem with CDK8-Mediator regulation of RNA polymerase II (Taatjes D. J. Trends Biochem Sci 35:315-22 (2010)). CDK9, another transcriptional CDK family member that has shared functions with CDK8 (Fryer C. J. et al., Mol Cell 16:509-20 (2004); Alarcon C. et al., Cell 139:757-69 (2009)), has also been shown to regulate ES cell pluripotency (Kaichi S. et al., J Cell Physiol 226:248-54 (2011)). And since both CDK8 and CDK9 have been found to phosphorylate RNA polymerase II in similar ways (Pinhero R. et al., Eur J Biochem 271:1004-14 (2004).), CDK8 and CDK9 may cooperate to modulate the transcription of ES cell-related genes, either in combination with or independently of MYC. Further, because MYC is not able to fully rescue the differentiation phenotype caused by CDK8 loss, further investigation is needed to identify MYC-independent mechanisms that CDK8 may be acting through to maintain tumors and stem cells in an undifferentiated state.

CDK8 inhibition in colon cancer cells leads to a significant decrease in the expression of ES cell-related genes, and these genes were particularly enriched for MYC target genes previously identified in ES cells. The subset of MYC target genes whose expression was CDK8 dependent was unique in its ability to predict tumor differentiation and clinical outcome. Specifically, increased expression of the CDK8-regulated MYC target genes singled out tumors that were poorly differentiated and were more prone to undergo rapid recurrence. This is in contrast to expression of the full set of MYC target genes, which were unable to identify these same tumors. These data suggest that the CDK8-regulated subset of MYC ES cell target genes are coordinately expressed in poorly differentiated, poor prognosis primary colon tumors. However it remains to be determined whether CDK8 is directly responsible for maintaining this coordinated expression.

In conclusion, convergent roles for CDK8 were defined regulating both tumor and ES cell differentiation states through regulating MYC. A CDK8-regulated MYC signature that was specifically expressed in poor prognosis colon tumors that were poorly differentiated was identified. Together these observations raise the possibility that the stem cell-like properties of cancer cells may be specifically inhibited by therapeutically targeting CDK8.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1: A method of screening for and/or identifying a CDK8 antagonist which promotes cell differentiation said method comprising: contacting a reference cell, wherein the reference cell is a stem cell and/or a cancer stem cell, with a CDK8 candidate antagonist, wherein the CDK8 candidate antagonist binds CDK8, and whereby differentiation of the reference cell into a differentiated cell identifies the CDK8 candidate antagonist as a CDK8 antagonist which promotes cell differentiation.

2: The method of claim 1, wherein the reference cell is a cancer stem cell.

3: The method of claim 2, wherein the differentiated cell is a goblet cell and/or enterocyte cell.

4: The method of claim 1, wherein the CDK8 candidate antagonist is an antibody, binding polypeptide, small molecule, or polynucleotide.

5: A method of inducing differentiation comprising contacting the cell with an effective amount of CDK8 antagonist.

6: The method of claim 5, wherein the cell is a stem cell.

7: The method of claim 5, wherein the cell is a cancer stem cell.

8: A method of treating a cancer cell, wherein the cancer cell differentially expresses one or more biomarkers of a CDK8 gene signature (e.g., compared to a reference sample, reference cell, reference tissue, control sample, control cell, control tissue, or internal control (e.g., housekeeping gene)), the method comprising providing an effective amount of a CDK8 antagonist.

9-19. (canceled)

20: The method of claim 1, wherein differential expression of one or more biomarkers of the CDK8 gene signature is elevated expression of one or more CDK8-induced biomarkers of the CDK8 gene signature and/or reduced expression of one or more CDK8-repressed biomarkers of the CDK8 gene signature.

21: (canceled)

22: The method of claim 8, wherein the one or more biomarkers of the CDK8 gene signature comprises one or more biomarkers of the CDK8 cancer cell gene signature.

23: The method of claim 22, wherein the one or more biomarkers of the CDK8 cancer cell gene signature comprises one or more genes listed in Table 2.

24: The method of claim 23, wherein the one or more genes listed in Table 2 comprises one or more ES cell-related genes, MYC ES target genes, p53 signalling genes, cell cycle genes, Wnt signalling genes, and/or SMAD/BMP signalling genes.

25: The method of claim 8, wherein the one or more biomarkers of the CDK8 gene signature comprises one or more biomarkers of the CDK8 embryonic stem cell gene signature.

26: The method of claim 8, wherein the one or more biomarkers of the CDK8 embryonic stem cell gene signature comprises one or more genes listed in Table 3.

27. (canceled)

28: The method of claim 8, wherein the CDK8 antagonist is an antibody, binding polypeptide, small molecule, or polynucleotide.

29: The method of claim 28, wherein the CDK8 antagonist is an antibody.

30: The method of claim 28, wherein the CDK8 antagonist is a small molecule.

31: The method of claim 29, wherein the small molecule is a small molecule kinase inhibitor.

32: The method of claim 30, wherein the small molecule kinase inhibitor is selected from the group consisting of flavopiridol, ABT-869, AST-487, BMS-387032/SNS032, BIRB-796, sorafenib, staurosporine, cortistatin, cortistatin A, and/or a steroidal alkaloid or derivative thereof.

33: The method of claim 28, wherein the CDK8 antagonist induces cell cycle arrest or is capable of promoting differentiation.

34: The method of claim 28, wherein the CDK8 antagonist is capable of promoting a change in cell fate and promoting differentiation is indicated by reduced expression of one or more CDK8-induced biomarkers of the CDK8 gene signature and/or elevated expression of one or more CDK8-reduced biomarkers of the CDK8 gene signature.


SEQUENCE LISTING (18 sequences)

SEQ ID NO: 1 - RNA, 19 nt, Artificial Sequence
  Description of Artificial Sequence Synthetic oligonucleotide
agccaagaggaaagauggg

SEQ ID NO: 2 - RNA, 18 nt, Artificial Sequence
  Description of Artificial Sequence Synthetic oligonucleotide
gcgaauuacucagaacag

SEQ ID NO: 3 - RNA, 19 nt, Artificial Sequence
  Description of Artificial Sequence Synthetic oligonucleotide
agguguuucugucucaugc

SEQ ID NO: 4 - RNA, 19 nt, Artificial Sequence
  Description of Artificial Sequence Synthetic oligonucleotide
uagaaggaacugggaucuc

SEQ ID NO: 5 - DNA, 21 bp, Artificial Sequence
  Description of Artificial Sequence Synthetic oligonucleotide
gaatggtgaagtcactattat

SEQ ID NO: 6 - DNA, 21 bp, Artificial Sequence
  Description of Artificial Sequence Synthetic oligonucleotide
caacaagatgaagagcaccaa

SEQ ID NO: 7 - DNA, 21 bp, Artificial Sequence
  Description of Artificial Sequence Synthetic oligonucleotide
cccgattatttaattcacctt

SEQ ID NO: 8 - DNA, 21 bp, Artificial Sequence
  Description of Artificial Sequence Synthetic oligonucleotide
cagggatttgaaacctgctaa

SEQ ID NO: 9 - DNA, 21 bp, Artificial Sequence
  Description of Artificial Sequence Synthetic oligonucleotide
gccagtgatttggaggtgaat

SEQ ID NO: 10 - DNA, 21 bp, Artificial Sequence
  Description of Artificial Sequence Synthetic oligonucleotide
cctctccctttgatgatccta

SEQ ID NO: 11 - DNA, 21 bp, Artificial Sequence
  Description of Artificial Sequence Synthetic oligonucleotide
ccgtgcgattaccaatgcaaa

SEQ ID NO: 12 - DNA, 21 bp, Artificial Sequence
  Description of Artificial Sequence Synthetic oligonucleotide
caaaactagtaatccttattt

SEQ ID NO: 13 - DNA, 21 bp, Artificial Sequence
  Description of Artificial Sequence Synthetic oligonucleotide
cccttacccaaaacgagaatt

SEQ ID NO: 14 - DNA, 20 bp, Artificial Sequence
  Description of Artificial Sequence Synthetic oligonucleotide
cccatctttcctcttggctt

SEQ ID NO: 15 - DNA, 20 bp, Artificial Sequence
  Description of Artificial Sequence Synthetic oligonucleotide
ctgttctgaggtaattcgct

SEQ ID NO: 16 - DNA, 21 bp, Artificial Sequence
  Description of Artificial Sequence Synthetic oligonucleotide
gcatgagacagaaacaccctt

SEQ ID NO: 17 - DNA, 20 bp, Artificial Sequence
  Description of Artificial Sequence Synthetic oligonucleotide
gagatcccagttccttctat

SEQ ID NO: 18 - RNA, 19 nt, Artificial Sequence
  Description of Artificial Sequence Synthetic oligonucleotide
guuuuugccgguugucaaa